(19) United States
(12) Patent Application Publication
(10) Pub. No.: US 2026/0015397 A1
(43) Pub. Date: Jan. 15, 2026

(54) CHIMERIC MEMBRANE-BOUND CYTOKINES INCORPORATING COSTIMULATORY ELEMENTS FOR ENHANCING ANTI-INFLAMMATORY FUNCTION

(71) Applicant: MIGAL GALILEE RESEARCH INSTITUTE LTD., Kiryat Shmona (IL)

(72) Inventors: Gideon GROSS, Moshav Almagor (IL); Hadas WEINSTEIN-MAROM, Kibbutz Dafna (IL); Dayana BLOKON-KOGAN, Kiryat Shmona (IL); Maya LEVI-MANN, Tiberias (IL)

(73) Assignee: MIGAL GALILEE RESEARCH INSTITUTE LTD., Kiryat Shmona (IL)

(21) Appl. No.: 19/100,725

(22) PCT Filed: Aug. 3, 2023
(86) PCT No.: PCT/IL2023/050812

§ 371 Date: Feb. 3, 2025

Related U.S. Application Data
(60) Provisional application No. 63/370,358, filed on Aug. 3, 2022.

(51) Int. Cl. C07K 14/54; A61K 40/11; A61K 40/35; C07K 14/705; C07K 14/74; C12N 5/0783

(52) U.S. Cl. CPC C07K 14/5428; A61K 40/11; A61K 40/35; C07K 14/70539; C07K 14/70578; C12N 5/0636; C07K 2319/03; C12N 2510/00

(57) ABSTRACT

Nucleic acid molecules comprising a sequence encoding a chimeric polypeptide comprising a cytokine region comprising at least one single-chain anti-inflammatory cytokine, a transmembrane region and a at least one signaling region comprising an element of a T cell costimulatory receptor of the tumor necrosis factor receptor (TNFR) family are provided. Expression vectors, polypeptides, cells, enriched populations and pharmaceutical compositions are also provided, as are methods for inducing immune suppression and treating a disease, disorder or condition characterized by excessive activity of the immune system.

### CROSS REFERENCE TO RELATED APPLICATIONS

This application claims the benefit of priority of U.S. Provisional Patent Application No. 63/370,358, filed Aug. 3, 2022, the contents of which are all incorporated herein by reference in their entirety.

### REFERENCE TO AN ELECTRONIC SEQUENCE LISTING

The contents of the electronic sequence listing (MIG-P-033-PCT_SQL.xml; Size: 153,398 bytes; and Date of Creation: Aug. 1, 2023) is herein incorporated by reference in its entirety.

### FIELD OF THE INVENTION

The invention relates to the field of genetically reprogrammed immune cells expressing membrane-bound IL10 fused with additional costimulatory elements and their use in increasing systemic immunosuppression and treating diseases manifested in excessive activity of the immune system.

### BACKGROUND

Harnessing CD4 regulatory T cells (Tregs) for suppressing local inflammation and restoring immunological balance holds great promise in the treatment of pathologies as diverse as autoimmune diseases, inflammatory bowel diseases, allergies, atherosclerosis, transplant rejection, graft-versus-host disease and more. However, Tregs, either natural (nTregs) or induced (iTregs), including type 1 regulatory T cells (Tr1 cells) form only a minor fraction in the entire human CD4 T cell population. Consequently, there is an urgent need for the development of Treg-based therapies designed for recruiting, inducing, or engineering autologous or allogeneic Tregs at adequate numbers and stable phenotype which are critical for clinical efficacy and safety of treatment.

An important subtype of iTregs, the type 1 or Tr1 cells are induced in the periphery in a TCR- and antigen-specific manner upon chronic exposure to antigen on dendritic cells in the presence of interleukin 10 (IL-10). Tr1 cells are characterized by a non-proliferative (anergic) state, high production of IL-10 and TGF-β and the ability to suppress effector T cells (Teffs) in a cell-to-cell contact-independent manner. A recent study demonstrated that the enforced constitutive expression of IL-10 in human CD4 T cells, accomplished by lentiviral transduction, was sufficient for endowing these cells with a particularly stable Tr1 phenotype in an autocrine fashion (Andolfi, et al., “Enforced IL-10 expression confers type 1 regulatory T cell (Tr1) phenotype and function to fuman CD4+ T cells”, 2012, Molecular Therapy, 20, 1778-1790). Although providing an elegant solution to de-novo generation of Tr1 cells, this protocol results in Tr1 cells that produce IL-10 constitutively, in an activation-independent manner. In the clinical setting this uncontrolled IL-10 secretion poses the risk of systemic and prolonged immune suppression, losing the intended antigen-or tissue-selectivity of the therapeutic effects exerted by the Tr1 cells.

There remains therefore a pressing need for efficient Treg- and in particular-efficient Tr1 immunotherapies for autoimmune disease and other autoimmune-related disorders.

### SUMMARY OF INVENTION

The present invention provides nucleic acid molecules comprising a sequence encoding a chimeric polypeptide comprising a cytokine region comprising at least one single-chain anti-inflammatory cytokine, a transmembrane region and a at least one signaling region comprising an element of a T cell costimulatory receptor of the tumor necrosis factor receptor (TNFR) family. Expression vectors, polypeptides, cells, enriched populations and pharmaceutical compositions are also provided, as are methods for inducing immune suppression and treating a disease, disorder or condition characterized by excessive activity of the immune system.

According to a first aspect, there is provided a nucleic acid molecule comprising a nucleotide sequence encoding a chimeric polypeptide comprising:
a. a cytokine region comprising at least one single-chain anti-inflammatory cytokine;
b. a transmembrane region; and
c. at least one signaling region comprising an element of a T cell costimulatory receptor of the tumor necrosis factor receptor (TNFR) family protein.

According to some embodiments, the single chain anti-inflammatory cytokine is a single chain interleukin 10 (IL-10).

According to some embodiments, the single chain IL-10 comprises SEQ ID NO: 1.

According to some embodiments, the cytokine region comprises a tandem repeat of the single chain anti-inflammatory cytokine, wherein the tandem repeats are linked by an amino acid linker.

According to some embodiments, the at least one single chain anti-inflammatory cytokine is a tandem repeat of single chain IL-10 linked by an amino acid linker.

According to some embodiments, the tandem repeat of single chain IL-10 comprises SEQ ID NO: 5.

According to some embodiments, the transmembrane region comprises a transmembrane domain of a single pass transmembrane protein, and wherein the protein is selected from an human leukocyte antigen A (HLA-A), an HLA-B, an HLA-C, CD40, Toll-like receptor 4 (TLR4), and CD28.

According to some embodiments, the transmembrane domain is a transmembrane domain of HLA-A or CD40.

According to some embodiments, the HLA-A is HLA-A2.

According to some embodiments, the HLA transmembrane domain comprises SEQ ID NO: 12, the CD40 transmembrane domain comprises SEQ ID NO: 11 or both.

According to some embodiments, the T cell costimulatory receptor of the TNFR family protein is selected from a CD40, CD27, 4-1BB (CD137), OX40 (CD134), herpesvirus entry mediator (HVEM/TNFRSF14), CD30, and glucocorticoid-induced TNFR-related protein (GITR).

According to some embodiments, the T cell costimulatory receptor of the TNFR family is CD40.

According to some embodiments, a signaling element of CD40 comprises SEQ ID NO: 14.

According to some embodiments, the signaling region further comprises at least one self-assembly domain.

According to some embodiments, the at least one self-assembly domain is a coiled coil domain from a yeast GCN4 leucine zipper DNA-binding motif.

According to some embodiments, the GCN4 coiled coil domain comprises SEQ ID NO: 31 or SEQ ID NO: 68.

According to some embodiments, the signaling region comprises a trimeric CD40 signaling element comprising SEQ ID NO: 13.

According to some embodiments, the polypeptide further comprises a membrane-proximal region selected from SEQ ID NO: 8, 9 and 10.

According to some embodiments, the regions are directly linked by amino acid linkers or peptide bonds.

According to some embodiments, the polypeptide further comprises a signal peptide.

According to some embodiments, the polypeptide comprises a sequence selected from SEQ ID NO: 15-30.

According to some embodiments, the polypeptide comprises a sequence selected from SEQ ID NO: 19-22 and 27-30.

According to some embodiments, the nucleic acid molecule comprises a nucleotide sequence selected from SEQ ID NO: 32-47.

According to another aspect, there is provided an expression vector, comprising a nucleic acid molecule of the invention operably linked to at least one transcription regulatory element.

According to some embodiments, the vector is an RNA or DNA expression vector.

According to some embodiments, the at least one transcription regulatory element is a T cell active promoter.

According to some embodiments, the promoter is selected from a nuclear factor of activated T-cells (NFAT)-responsive promoter, an NF-KB promoter, an IL-2 promoter, and an IL-2 receptor (IL-2R) promoter.

According to some embodiments, the vector is a retroviral vector.

According to another aspect, there is provided a polypeptide encoded by a nucleic acid molecule of the invention.

According to another aspect, there is provided a cell comprising a nucleic acid molecule of the invention, a vector of the invention or a polypeptide of the invention.

According to some embodiments, the cell is a T cell.

According to some embodiments, the cell is a CD4 positive T cell.

According to some embodiments, the cell comprises increased secretion of IL-10 or interferon gamma (IFNg) as compared to a cell that does not comprises the nucleic acid molecule, vector or polypeptide.

According to another aspect, there is provided an enriched population of CD4 T cells, wherein at least 10% of cells in the population comprise a nucleic acid molecule of the invention, a vector of the invention or the polypeptide of the invention.

According to another aspect, there is provided a method of converting a CD4 positive T cell into a regulatory T cell (Treg), the method comprising introducing into the CD4 positive T cell a nucleic acid molecule of the invention, a vector of the invention or a polypeptide of the invention, thereby converting a CD4 positive T cell into a Treg.

According to some embodiments, the CD4 positive T cell is a primary cell isolated from peripheral blood.

According to another aspect, there is provided an artificial Treg cell produced by a method of the invention.

According to another aspect, there is provided a pharmaceutical composition comprising a nucleic acid molecule of the invention, a vector of the invention, the polypeptide of the invention, a cell of the invention, the enriched population of the invention or the artificial Treg of the invention and a pharmaceutically acceptable carrier excipient or adjuvant.

According to another aspect, there is provided a method for inducing immune suppression in a subject in need thereof, the method comprising administering to the subject a pharmaceutical composition of the invention, thereby inducing immune suppression.

According to another aspect, there is provided a method for treating a disease, disorder or condition characterized by excessive activity of the immune system or treatable by immune suppression in a subject in need thereof, the method comprising administering to the subject a pharmaceutical composition of the invention, thereby treating a disease, disorder or condition.

According to some embodiments, the disease, disorder or condition is selected from an autoimmune and an inflammatory disease, disorder or condition.

According to some embodiments, the disease, disorder or condition is selected from transplant rejection, cardiovascular disease, obesity, systemic inflammation, celiac disease, dermatomyositis, Graves' disease, Addison's disease, Hashimoto disease, Multiple sclerosis, Crohn's disease, colitis, myasthenia gravis, pernicious anemia, arthritis, Sjogren syndrome, lupus, diabetes, pemphigus vulgaris, pemphigus follicularis, membranous nephropathy, sarcoidosis, vasculitis, atherosclerosis, granulomatosis, spondyloarthropathy and cytokine storm.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1. Eight memIL-10-CD40 constructs. Schematics of eight memIL-10-CD40 constructs of the invention are shown. The eight are: 2101: CFR-CD40Tm-CD40GCN4-; 2102: CFR-CD40Tm-CD40GCN4+; 2103: CFR-HLATm-CD40GCN4-; 2104: CFR-HLATm-CD40GCN4+; 2105: HLA-A2-CD40Tm-CD40GCN4-; 2106: HLA-A2-CD40Tm-CD40GCN4+; 2107: HLA-A2-HLATm-CD40GCN4-; and 2108: HLA-A2-HLATm CD40GCN4+.

FIGS. 2A-C: Surface expression of memIL-10-CD40 in CD4 cells. (2A) Flow cytometry data for the surface expression of memIL-10-CD40 constructs in CD4 cells, 4 hours post-mRNA transfection. (2B-C) Histograms showing upregulation of the Tr1 markers (2B) LAG3 and (2C) PD-1 in mRNA-transfected CD4+ T cells of a healthy donor. CD4+ T Cells were transfected with 10 μg of the indicated mRNAs and stained for cell surface LAG-3 and PD-1, using an eFlour710-conjugated anti-human CD223 (LAG3) mAb and an AF700PE-conjugated anti-human PD-1 mAb.

FIGS. 3A-3F: ELISA test results. (3A) Comparison of IFN-γ secretion with and without CAR activation. (3B) IFN-γ secretion without CAR activation. (3C) IFN-γ secretion after CAR activation. (3D) Comparison of IL-10 secretion with and without CAR activation. (3E) IL-10 secretion without CAR activation. (3F) IL-10 secretion after CAR activation.

FIG. 4: Flow Analysis results for CD4 and HLA-A2 demonstrating the presence of a pure CD4 T cell population.

FIGS. 5A-5F: ELISA test results-IFN-γ and IL-10 secretion: (5A) Comparison of IFN-γ secretion with and without TCR activation. (5B) IFN-γ secretion without TCR activation. (5C) IFN-γ secretion after TCR activation. (5D) Comparison of IL-10 secretion with and without TCR activation. (5E) IL-10 secretion without TCR activation. (5F) IL-10 secretion after TCR activation.

FIG. 6: Transduction results. Flow analysis of IL-10 surface expression after 5 days, 11 days and 18 days. Below is a line graph of cell viability across the same time period.

### DETAILED DESCRIPTION

The inventors have developed an “all-in-one” IL-10-CD40 cassette designed to trigger simultaneous activation of at least two T cell costimulatory pathways through one genetically engineered membrane molecule. The cassette includes a membrane-bound IL-10 (memIL-10) in a tandem arrangement at least with a CD40 or a constitutively active CD40 (caCD40) signaling domain. The memIL-10 generally comprises a single-chain IL-10 (scIL-10) in tandem, a linker, a bridge element, and a trans-membrane domain.

Specifically, the inventors present synthetic surface receptors made of a membrane-bound IL-10 (memIL-10), fused with a CD40 or a constitutively active CD40 and/or TLR4 signaling domain (memIL-10-CD40 or memIL-10-TLR4-CD40) as ligand-independent costimulatory receptors to improve CD4 T cell reactivity.

The list of the constructs prepared and the elements comprising these constructs is detailed in Tables 1 and hereinbelow.

TABLE 1
Summary of constructs - All-in-one: scIL-10 + CD40

| Construct no. | SEQ ID | scCyt | Linker | Bridge | TM | IR |
|---|---|---|---|---|---|---|
| 2101 | 15 | IL-10 | S-Linker | CFR | CD40 | CD40 |
| 2102 | 16 | IL-10 | S-Linker | CFR | CD40 | GCN4-CD40 trimer |
| 2103 | 17 | IL-10 | S-Linker | CFR | HLA | CD40 |
| 2104 | 18 | IL-10 | S-Linker | CFR | HLA | GCN4-CD40 trimer |
| 2105 | 19 | IL-10 | S-Linker | HLA-A2 | CD40 | CD40 |
| 2106 | 20 | IL-10 | S-Linker | HLA-A2 | CD40 | GCN4-CD40 trimer |
| 2107 | 21 | IL-10 | S-Linker | HLA-A2 | HLA | CD40 |
| 2108 | 22 | IL-10 | S-Linker | HLA-A2 | HLA | GCN4-CD40 trimer |

In a first aspect, there is provided a nucleic acid molecule comprising a nucleotide sequence encoding a chimeric polypeptide comprising: a cytokine region, a transmembrane region and at least one signaling region.

In one aspect, the present invention provides a nucleic acid molecule comprising a nucleotide sequence encoding a polypeptide comprising a chimeric anti-inflammatory cytokine including: at least one single-chain anti-inflammatory cytokine linked to a transmembrane region which is linked to a cytoplasmic region including at least one signaling element of a T cell costimulatory receptor of the tumor necrosis factor receptor (TNFR) family optionally linked to at least one self-assembly domain.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The terms “nucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, lncRNA, circulating nucleic acids, fragments of genomic DNA or RNA, degraded nucleic acids, ribozymes, viral RNA or DNA, nucleic acids of infectious origin, amplification products, modified nucleic acids, plasmidical or organellar nucleic acids and artificial nucleic acids such as oligonucleotides. In some embodiments, the nucleic acid molecule is DNA. In some embodiments, the nucleic acid molecule is devoid of introns. It is noted that the DNA sequences which encode the single-chain cytokine may be based on genomic sequences or on RNA sequences (e.g., excluding introns).

In some embodiments, the nucleic acid molecule is a vector. In some embodiments, the vector is an expression vector. In some embodiments, the expression vector is a mammalian expression vector. In some embodiments, the vector is a DNA vector. In some embodiments, the vector is an RNA vector. In some embodiments, the nucleic acid molecule is for use in a method of the invention.

In a further aspect, the present invention provides a vector comprising the nucleic acid molecule of any one of the herein provided embodiments. The vector may be a retroviral vector, a lentiviral vector, a herpes viral vector, an adeno-associated viral vector, a transposon vector and an adenoviral vector. In some embodiments, the vector is a retroviral vector.

As used herein, the term “expression” refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide). Expressing a gene/open reading frame within a cell is well known to one skilled in the art. It can be carried out by, among many methods, transfection, viral infection, or direct alteration of the cell's genome. In some embodiments, the gene is in an expression vector such as plasmid or viral vector.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoter may be a viral promoter.

In some embodiments, the vector is introduced into the cell by standard methods including electroporation (e.g., as described in From et al., Proc. Natl. Acad. Sci. USA 82, 5824 (1985)),Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. In some embodiments, the promoter is a tissue specific promoter. In some embodiments, the promoter is a cell or cell type specific promoter. In some embodiments, the cell is the target cell. In some embodiments, the cell is an immune cell. In some embodiments, the cell is a T cell. In some embodiments, the promoter is a T cell specific promoter. Examples of T cell specific promoters include for example the CD3 promoter.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (+), pGL3, pZeoSV2 (+), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

A person with skill in the art will appreciate that a gene/open reading frame can also be expressed from a nucleic acid construct administered to the individual employing any suitable mode of administration, described hereinabove (i.e., in vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex vivo gene therapy).

In some embodiments, the nucleic acid molecule comprises an open reading frame encoding the chimeric polypeptide. In some embodiments, the open reading frame is operably linked to at least one transcription regulatory element. In some embodiments, the transcription regulatory element is active in a target cell. In some embodiments, the regulatory element is a promoter. In some embodiments, the nucleic acid molecule comprises a promoter. In some embodiments, the promoter is a heterologous promoter. In some embodiments, the promoter is a constitutive promoter. In some embodiments, the promoter is an inducible promoter. In some embodiments, the promoter is a viral promoter. In some embodiments, the promoter is a human promoter. In some embodiments, the promoter is active in a target cell. In some embodiments, the target cell is an immune cell. In some embodiments, the immune cell is a lymphocyte. In some embodiments, the immune cell is a T cell. In some embodiments, the promoter is a T cell active promoter. In some embodiments, the T cell is a CD4 positive T cell. In some embodiments, the T cell is a helper T cell. In some embodiments, the T cell is a not a T regulatory cell (Treg). In some embodiments, the promoter is a T cell promoter. In some embodiments, the promoter is a target cell promoter. In some embodiments, the promoter is T cell specific. In some embodiments, the promoter is selected from a nuclear factor of activated T-cells (NFAT)-responsive promoter, an NF-KB promoter, an IL-2 promoter, and an IL-2 receptor (IL-2R) promoter. In some embodiments, the nucleic acid is DNA, and the nucleotide sequence further includes at least one T cell activation control element, such as a nuclear factor of activated T-cells (NFAT)-responsive promoter, an NF-KB promoter, an IL-2 promoter, or an IL-2 receptor (IL-2R) promoter, which controls the expression of the polypeptide. A T cell activation control element is a promoter or another nucleic acid element involved in transcription regulation, that upregulates transcription when the T cell is activated, such as nuclear factor of activated T-cells (NFAT)-responsive promoter, an NF-KB promoter, an IL-2 promoter, or an IL-2 receptor (IL-2R) promoter.

As used herein, the term “operably linked” is intended to mean that the nucleotide sequence of interest is linked to the regulatory element or elements in a manner that allows for expression of the nucleotide sequence (e.g., in an in vitro transcription/translation system or in a host cell when the nucleic acid molecule is introduced into the host cell).

In some embodiments, the polypeptide comprises a cytokine region. In some embodiments, the nucleotide sequence comprises a sequence that encodes the cytokine region. In some embodiments, cytokine region comprises at least one cytokine. In some embodiments, the cytokine is an anti-inflammatory cytokine. In some embodiments, the cytokine is interleukin 10 (IL-10). In some embodiments, the cytokine is a single-chain cytokine. In some embodiments, the IL-10 is a single-chain IL-10. In some embodiments, single-chain IL-10 is a monomer. In some embodiments, single-chain IL-10 is monomeric IL-10. In some embodiments, monomeric IL-10 comprises the amino acid sequence SPGQGTQSENSCTHFPGNLPNMLRDLRDAFSRVKTFFQMKDQLDNLLLKESLLEDFKG YLGCQALSEMIQFYLEEVMPQAENQDPDIKAHVNSLGENLKTLRLRLRRCHRFLPCEN KSKAVEQVKNAFNKLQEKGIYKAMSEFDIFINYIEAYMTMKIRN (SEQ ID NO: 1). In some embodiments, monomeric IL-10 consists of SEQ ID NO: 1. In some embodiments, SEQ ID NO: 1 is encoded by a nucleotide sequence comprising SEQ ID NO: 48. In some embodiments, SEQ ID NO: 1 is encoded by a nucleotide sequence consisting of SEQ ID NO: 48. It will be understood by a skilled artisan that any synonymous mutations can be made within SEQ ID NO: 48 and it will still encode SEQ ID NO: 1 and so all such nucleotide sequences are also contemplated.

The term “single-chain cytokine” as used herein is intended to encompass any cytokine contained in a single polypeptide chain. This term encompasses both natural cytokine sequences in which the sequence of the single-chain cytokine is the same as the natural cytokine sequence, e.g. in cases where the cytokine naturally includes only a single polypeptide, as well as processed or synthetic cytokines, e.g. when the cytokine naturally includes more than a single chain, where the separate chains have been fused into a single-chain recombinant cytokine, similar to the concept of an scFv (single-chain variable fragment) of an immunoglobulin. Examples for the preparation of single-chain cytokines can be found in Chmielewski et al., 2011, “IL-12 release by engineered T cells expressing chimeric antigen receptors can effectively Muster an antigen-independent macrophage response on tumor cells that have shut down tumor antigen expression”, Cancer Res. 2011 Sep. 1;71 (17): 5697-706, Weinstein-Marom et al., 2016, “Membrane-attached Cytokines Expressed by mRNA Electroporation Act as Potent T-Cell Adjuvants”, J Immunother. 2016 February-Mar;39 (2): 60-70 and Chinnasamy et al., 2012, “T Cells That Target Carcinoembryonic Antigen Eradicate Orthotopic Pancreatic Carcinomas Without Inducing Autoimmune Colitis in Mice”, Gastroenterologyl43,1095-107.e2, the contents of which are all hereby incorporated by reference in their entirety.

In some embodiments, the polypeptide further comprises a signal peptide. In some embodiments, the nucleic acid molecule comprises a nucleotide sequence that encodes the signal peptide. In some embodiments, the signal peptide is a human signal peptide. In some embodiments, the signal peptide is a cytokine signal peptide. In some embodiments, the leader peptide is the cytokine original signal peptide. In some embodiments, the signal peptide is an IL-10 signal peptide. It will be understood by a skilled artisan that the signal peptide is merely needed for targeting to the ER and expression of the polypeptide of the invention within a membrane. The specific signal peptide used is immaterial and any signal peptide may be employed. It will further be understood that while the nucleic acid molecule will include a sequence encoding the signal peptide, the amino acid sequence of the peptide will be cleaved from the polypeptide in its final form. Thus, amino acid sequences comprising the signal peptide and without the signal peptide are both contemplated.

In another aspect, the present invention provides a nucleic acid molecule comprising a nucleotide sequence encoding a polypeptide comprising a membrane-bound IL-10, which includes a single-chain IL-10 linked at the amino-terminal end to an immunoglobulin V-region leader peptide, such as a leader peptide of VH, Vκ, or Vλ. In some embodiments, the signal peptide is an immunoglobulin signal peptide. As used herein, “leader peptide” and “signal peptide are used herein interchangeably. In some embodiments, the signal peptide is selected from a VH and VL signal peptide. In some embodiments, VL is selected from Vkappa (Vκ) and Vlambda (Vλ). In some embodiments, the VL leader peptide is MAWTVLLLGLLSHCTGSVT (SEQ ID NO: 82). In some embodiments, the signal peptide comprises SEQ ID NO: 82.

In some embodiments, the signal peptide comprises MHSSALLCCLVLLTGVRA (SEQ ID NO: 4). In some embodiments, the signal peptide consists of SEQ ID NO: 4. In some embodiments, the signal peptide is encoded by a nucleotide sequence comprising SEQ ID NO: 49. In some embodiments, the signal peptide is encoded by a nucleotide sequence consisting of SEQ ID NO: 49.

In some embodiments, the cytokine region comprises at least two copies of the cytokine. In some embodiments, the cytokine region comprises at least two copies of the single-chain cytokine. In some embodiments, the cytokine region comprises at least two copies of the cytokine monomer. In some embodiments, at least two copies is two copies. In some embodiments, the cytokine region comprises repeats of the cytokine. In some embodiments, repeats are two repeats. In some embodiments, repeats are tandem repeats. In some embodiments, repeats are two copies. In some embodiments, the repeats are the same. In some embodiments, the repeats are identical. In some embodiments, the repeats are different. In some embodiments, the repeats are tandem repeats of single chain IL-10. In some embodiments, the repeats are tandem repeats of monomeric IL-10. In some embodiments, the repeats are repeats of SEQ ID NO: 1. In some embodiments, the cytokine region comprises two copies of SEQ ID NO: 1.

In some embodiments, the repeats are linked by a linker. In some embodiments, a linker is a spacer. In some embodiments, the linker is an amino acid linker. In some embodiments, the linker comprises a peptide bond. In some embodiments, the linker comprises at least 1, 2, 3, 4, 5, 6, 7, 8, 9, 10, 11, 12, 13, 14, 15, 16, 17, 18, 19 or 20 amino acids. Each possibility represents a separate embodiment of the invention. In some embodiments, the linker comprises at least 10 amino acids. In some embodiments, the linker comprises at least 15 amino acids. In some embodiments, the linker comprises at least 18 amino acids. In some embodiments, the linker consists of 1, 2, 3, 4, 5, 6, 7, 8, 9, 10, 11, 12, 13, 14, 15, 16, 17, 18, 19 or 20 amino acids. Each possibility represents a separate embodiment of the invention. In some embodiments, the linker is a flexible linker. In some embodiments, the linker comprises the amino acid sequence GSTSGSGKPGSGEGSTKG (SEQ ID NO: 3). In some embodiments the linker consists of SEQ ID NO: 3. In some embodiments, the linker is a GS linker. In some embodiments, the linker is a GGGGS (SEQ ID NO: 50) linker. In some embodiments, the linker comprises GGGGSGGGSGGGS (SEQ ID NO: 7). In some embodiments, the linker consists of SEQ ID NO: 7. In some embodiments, the linker comprises GGGGSGGGGSGGGGSGGGGSGGGGSGGGGSAS (SEQ ID NO: 51). In some embodiments, the linker consists of SEQ ID NO: 51. In some embodiments, SEQ ID NO: 7 is encoded by a nucleotide sequence comprising SEQ ID NO: 52. In some embodiments, SEQ ID NO: 7 is encoded by a nucleotide sequence consisting of SEQ ID NO: 52. In some embodiments, SEQ ID NO: 51 is encoded by a nucleotide sequence comprising SEQ ID NO: 53. In some embodiments, SEQ ID NO: 51 is encoded by a nucleotide sequence consisting of SEQ ID NO: 53. In some embodiments, SEQ ID NO: 3 is encoded by a nucleotide sequence comprising SEQ ID NO: 54. In some embodiments, SEQ ID NO: 3 is encoded by a nucleotide sequence consisting of SEQ ID NO: 54.

In some embodiments, the tandem repeat comprises the amino acid sequence SPGQGTQSENSCTHFPGNLPNMLRDLRDAFSRVKTFFQMKDQLDNLLLKESLLEDFKG YLGCQALSEMIQFYLEEVMPQAENQDPDIKAHVNSLGENLKTLRLRLRRCHRFLPCEN KSKAVEQVKNAFNKLQEKGIYKAMSEFDIFINYIEAYMTMKIRNGSTSGSGKPGSGEGS TKGSPGQGTQSENSCTHFPGNLPNMLRDLRDAFSRVKTFFQMKDQLDNLLLKESLLED FKGYLGCQALSEMIQFYLEEVMPQAENQDPDIKAHVNSLGENLKTLRLRLRRCHRFLP CENKSKAVEQVKNAFNKLQEKGIYKAMSEFDIFINYIEAYMTMKIRN (SEQ ID NO: 5).

In some embodiments, the tandem repeat consists of SEQ ID NO: 5. In some embodiments, the cytokine region comprises SEQ ID NO: 5. In some embodiments, the cytokine region consists of SEQ ID NO: 5. In some embodiments, SEQ ID NO: 5 is encoded by a nucleotide sequence comprising SEQ ID NO: 55. In some embodiments, SEQ ID NO: 5 is encoded by a nucleotide sequence consisting of SEQ ID NO: 55.

In some embodiments, the tandem repeat comprises a signal peptide and comprises the amino acid sequence MHSSALLCCLVLLTGVRASPGQGTQSENSCTHFPGNLPNMLRDLRDAFSRVKTFFQMK DQLDNLLLKESLLEDFKGYLGCQALSEMIQFYLEEVMPQAENQDPDIKAHVNSLGENL KTLRLRLRRCHRFLPCENKSKAVEQVKNAFNKLQEKGIYKAMSEFDIFINYIEAYMTM KIRNGSTSGSGKPGSGEGSTKGSPGQGTQSENSCTHFPGNLPNMLRDLRDAFSRVKTFF QMKDQLDNLLLKESLLEDFKGYLGCQALSEMIQFYLEEVMPQAENQDPDIKAHVNSL GENLKTLRLRLRRCHRFLPCENKSKAVEQVKNAFNKLQEKGIYKAMSEFDIFINYIEAY MTMKIRN (SEQ ID NO: 2). In some embodiments, the tandem repeat consists of SEQ ID NO: 2. In some embodiments, the cytokine region comprises a signal peptide and comprises SEQ ID NO: 2. In some embodiments, the cytokine region comprises a signal peptide and consists of SEQ ID NO: 2. In some embodiments, SEQ ID NO: 2 is encoded by a nucleotide sequence comprising SEQ ID NO: 56. In some embodiments, SEQ ID NO: 2 is encoded by a nucleotide sequence consisting of SEQ ID NO: 56.

In some embodiments, the polypeptide comprises a transmembrane region. In some embodiments, a transmembrane region is a highly hydrophobic region. In some embodiments, a transmembrane region is a hydrophobic region. In some embodiments, highly hydrophobic comprises at least 10, 15, 20, 25, 30, 35, 40, 45, 50, 55, 60, 65, 70, 75, 80, 85, 90, 92, 95, 97, 99 or 100% of the amino acid residues in the region being hydrophobic residues. Each possibility represents a separate embodiment of the invention. In some embodiments, highly hydrophobic comprises 100% of the amino acid residues in the region being hydrophobic residues. In some embodiments, highly hydrophobic comprises at least 90% of the amino acid residues in the region being hydrophobic residues. In some embodiments, highly hydrophobic comprises at least 95% of the amino acid residues in the region being hydrophobic residues. In some embodiments, highly hydrophobic comprises at most 1, 2, 3, 4 or 5 non-hydrophobic residues. Each possibility represents a separate embodiment of the invention. n some embodiments, highly hydrophobic comprises at most 1 non-hydrophobic residue. In some embodiments, a hydrophobic residue comprises a hydrophobic side chain. It is well known in the art that there are nine hydrophobic amino acids and they are: glycine, alanine, valine, leucine, isoleucine, proline, phenylalanine, methionine and tryptophan. A hydrophobic region (e.g., a transmembrane region) will be made up primarily, or entirely of these amino acids. In some embodiments, a transmembrane region is a region configured to insert into a lipid membrane. In some embodiments, a lipid membrane is a lipid bilayer.

In some embodiments, the transmembrane region comprises a transmembrane domain of a transmembrane protein. In some embodiments, a transmembrane protein is an integral membrane protein. In some embodiments, the region consists of the transmembrane domain. In some embodiments, the transmembrane protein is a single pass transmembrane protein. In some embodiments, the transmembrane region is selected from a transmembrane region of a transmembrane region of the heavy chain of a human MHC class I molecule selected from an HLA-A, HLA-B and HLA-C, preferably HLA-A2; a transmembrane region of CD40; a toll-like receptor (TLR) such as TLR4; and a transmembrane region of human CD28. In some embodiments, the protein is selected from human leukocyte antigen (HLA), CD40, Toll-like receptor 4 (TLR4) and CD28. In some embodiments, HLA is selected from HLA-A, HLA-b, and HLA-C. In some embodiments, the transmembrane domain is the transmembrane domain of HLA. In some embodiments, HLA is HLA-A.

In some embodiments, the transmembrane domain of HLA comprises VGIIAGLVLFGAVITGAVVAAVMW (SEQ ID NO: 12). In some embodiments, the transmembrane domain of HLA consists of SEQ ID NO: 12. In some embodiments, the transmembrane region comprises SEQ ID NO: 12. In some embodiments, the transmembrane region consists of SEQ ID NO: 12. In some embodiments, SEQ ID NO: 12 is encoded by a nucleotide sequence comprising SEQ ID NO: 57. In some embodiments, SEQ ID NO: 12 is encoded by a nucleotide sequence consisting of SEQ ID NO: 57.

In some embodiments, the transmembrane domain of CD40 comprises ALVVIPIIFGILFAILLVLVFI (SEQ ID NO: 11). In some embodiments, the transmembrane domain of CD40 consists of SEQ ID NO: 11. In some embodiments, the transmembrane region comprises SEQ ID NO: 11. In some embodiments, the transmembrane region consists of SEQ ID NO: 11. In some embodiments, SEQ ID NO: 11 is encoded by a nucleotide sequence comprising SEQ ID NO: 58. In some embodiments, SEQ ID NO: 11 is encoded by a nucleotide sequence consisting of SEQ ID NO: 58.

In some embodiments, the at least one cytokine is linked to the transmembrane region by at least one of (i) a flexible linker; (ii) a membrane-proximal region of the ectodomain of an integral membrane protein; and (iii) a carboxy-terminal flanking cysteine-rich region (CFR) of a leucine-rich repeats (LRR) motif-containing protein. In some embodiments, the at least one cytokine is linked to the transmembrane region by a flexible linker, and a membrane-proximal amino region of the ectodomain of an integral membrane protein or a carboxy-terminal flanking cysteine-rich region (CFR) of a leucine-rich repeats (LRR) motif-containing protein. In some embodiments, the at least one cytokine is linked to the transmembrane region by a flexible linker, and a membrane-proximal amino region of the ectodomain of an HLA-A2 heavy chain or a CFR of TLR4.

In some embodiments, the polypeptide further comprises a membrane-proximal region. In some embodiments, membrane-proximal is transmembrane region proximal. In some embodiments, the proximal region is an ectodomain. In some embodiments, the ectodomain is of an integral membrane protein. In some embodiments, the proximal region is a bridge region. In some embodiments, the protein is HLA. In some embodiments, HLA is HLA-A2. In some embodiments, the HLA bridge comprises SSQPTIPI (SEQ ID NO: 8). In some embodiments, the HLA bridge consists of SEQ ID NO: 8. In some embodiments, the membrane proximal region comprises SEQ ID NO: 8. In some embodiments, the membrane proximal region consists of SEQ ID NO: 8. In some embodiments, SEQ ID NO: 8 is encoded by a nucleotide sequence comprising SEQ ID NO: 59. In some embodiments, SEQ ID NO: 8 is encoded by a nucleotide sequence consisting of SEQ ID NO: 59. In some embodiments, the HLA bridge comprises LRWEPSSQPTIPI (SEQ ID NO: 10). In some embodiments, the HLA bridge consists of SEQ ID NO: 10. In some embodiments, the membrane proximal region comprises SEQ ID NO: 10. In some embodiments, the membrane proximal region consists of SEQ ID NO: 10. In some embodiments, SEQ ID NO: 10 is encoded by a nucleotide sequence comprising SEQ ID NO: 60. In some embodiments, SEQ ID NO: 10 is encoded by a nucleotide sequence consisting of SEQ ID NO: 60. In some embodiments, the proximal region is a CFR. In some embodiments, the CFR is a CFR bridge. In some embodiments, the carboxy-terminal flanking cysteine-rich region (CFR) is that of a TLR. In some embodiments, the carboxy-terminal flanking cysteine-rich region (CFR) is that of TLR4. In some embodiments, the CFR comprises PVLSLNITCQMNK (SEQ ID NO: 9). In some embodiments, the CFR consists of SEQ ID NO: 9. In some embodiments, the membrane proximal region comprises SEQ ID NO: 9. In some embodiments, the membrane proximal region consists of SEQ ID NO: 9. In some embodiments, SEQ ID NO: 9 is encoded by a nucleotide sequence comprising SEQ ID NO: 61. In some embodiments, SEQ ID NO: 9 is encoded by a nucleotide sequence consisting of SEQ ID NO: 61. In some embodiments, the membrane proximal region is selected from SEQ ID NO: 8, 9 and 10. In some embodiments, the membrane proximal region is selected from SEQ ID NO: 9 and 10.

In some embodiments, the integral membrane protein is a type I integral membrane protein. In some embodiments, the integral membrane protein is a type III integral membrane protein. In some embodiments, the integral membrane protein is an MHC heavy chain protein. In some embodiments, the integral membrane protein is a heavy chain of an HLA class I protein. In some embodiments, the integral membrane protein is a heavy chain of an HLA-A2 protein.

When the integral membrane protein is an HLA class I protein (such as HLA-A2), the membrane-proximal region of the ectodomain of the integral membrane protein is termed “HLA-bridge”.

In some embodiments, the polypeptide comprises membranal IL-10. In some embodiments, the cytokine region and transmembrane domain comprise membranal IL-10. In some embodiments, the cytokine region and transmembrane domain consists of membranal IL-10. In some embodiments, membranal IL-10 comprises MHSSALLCCLVLLTGVRASPGQGTQSENSCTHFPGNLPNMLRDLRDAFSRVKTFFQMK DQLDNLLLKESLLEDFKGYLGCQALSEMIQFYLEEVMPQAENQDPDIKAHVNSLGENL KTLRLRLRRCHRFLPCENKSKAVEQVKNAFNKLQEKGIYKAMSEFDIFINYIEAYMTM KIRNGSTSGSGKPGSGEGSTKGSPGQGTQSENSCTHFPGNLPNMLRDLRDAFSRVKTFF QMKDQLDNLLLKESLLEDFKGYLGCQALSEMIQFYLEEVMPQAENQDPDIKAHVNSL GENLKTLRLRLRRCHRFLPCENKSKAVEQVKNAFNKLQEKGIYKAMSEFDIFINYIEAY MTMKIRNGGGGSGGGSGGGSSSQPTIPIVGIIAGLVLFGAVITGAVVAAVMWRRKSSD RKGGSYSQAASSDSAQGSDVSLTACKV (SEQ ID NO: 6). In some embodiments, membranal IL-10 consists of SEQ ID NO: 6. In some embodiments, SEQ ID NO: 6 is encoded by a nucleotide sequence comprising SEQ ID NO: 62. In some embodiments, SEQ ID NO: 6 is encoded by a nucleotide sequence consisting of SEQ ID NO: 62. It will be understood that SEQ ID NO: 6 can also lack the signal peptide or contain a different signal peptide and so too SEQ ID NO: 62 can lack the sequence encoding the signal peptide or include a sequence encoding a different signal peptide.

In some embodiments, the cytokine region and the membrane proximal region are an extracellular region. In some embodiments, an extracellular region is a region N-terminal to the transmembrane region. In some embodiments, an extracellular region is a region that when the polypeptide is inserted into a plasma membrane of a cell is found extracellular to the cell. In some embodiments, the extracellular region comprises SEQ ID NO: 63. In some embodiments, the extracellular region consists of SEQ ID NO: 63. In some embodiments, SEQ ID NO: 63 is encoded by a nucleotide sequence comprising SEQ ID NO: 64. In some embodiments, SEQ ID NO: 63 is encoded by a nucleotide sequence consisting of SEQ ID NO: 64. In some embodiments, the extracellular region comprises SEQ ID NO: 65. In some embodiments, the extracellular region consists of SEQ ID NO: 65. In some embodiments, SEQ ID NO: 65 is encoded by a nucleotide sequence comprising SEQ ID NO: 66. In some embodiments, SEQ ID NO: 65 is encoded by a nucleotide sequence consisting of SEQ ID NO: 66.

In some embodiments, the signaling region is an intracellular region. The terms “intracellular region” (IR) and “cytoplasmic region” are used herein interchangeably. In some embodiments, an intracellular region is a region C-terminal to the transmembrane region. In some embodiments, an intracellular region is a region that when the polypeptide is inserted into a plasma membrane of a cell is found intracellular to the cell. In some embodiments, intracellular is in the cytoplasm. In some embodiments, the signaling region comprises a signaling domain. In some embodiments, the signaling domain is an element of a T cell costimulatory receptor of the tumor necrosis factor receptor (TNFR) family. In some embodiments, the element is a signaling element. In some embodiments, the element is a portion of the intracellular domain of the TNFR family protein. In some embodiments, the element comprises a portion of the intracellular domain of the TNFR family protein. In some embodiments, the element consists of a portion of the intracellular domain of the TNFR family protein. In some embodiments, the portion is the entire intracellular domain. The term “signaling element” as used herein refers to a functional cytoplasmic domain of the indicated protein, which participates in signal transduction.

In some embodiments, the cytoplasmic region of the chimeric anti-inflammatory cytokine comprises more than one signaling element, and the signaling elements are linked to each other by at least one flexible linker. In some embodiments, the cytoplasmic region further includes at least one toll/interleukin-1 receptor (TIR) signaling element, such as a TIR signaling element from a toll-like receptor such as TLR4, an Interleukin-1 receptor, or a cytosolic adaptor protein such as MyD88.

In some embodiments, the T cell costimulatory receptor of the TNFR family protein is selected from CD40, CD27, Tumor necrosis factor ligand superfamily member 9 (TNFRSF9/4-1BB/CD137), Tumor necrosis factor receptor superfamily, member 4 (TNFRSF4/OX40/CD134), herpesvirus entry mediator (HVEM/TNFRSF14), CD30, and glucocorticoid-induced TNFR-related protein (GITR). In some embodiments, the T cell costimulatory receptor of the TNFR family protein is CD40. In some embodiments, the CD40 is CD40 IR. In some embodiments, the signaling domain is the CD40 intracellular domain. In some embodiments, the CD40 signaling element comprises KKVAKKPTNKAPHPKQEPQEINFPDDLPGSNTAAPVQETLHGCQPVTQEDGKESRISV QERQ (SEQ ID NO: 14). In some embodiments, the CD40 signaling element consists of SEQ ID NO: 14. In some embodiments, the element of a T cell costimulatory receptor of TNFR family protein comprises SEQ ID NO: 14. In some embodiments, the element of a T cell costimulatory receptor of TNFR family protein consists of SEQ ID NO: 14. In some embodiments, the signaling region comprises SEQ ID NO: 14. In some embodiments, the signaling region consists of SEQ ID NO: 14. In some embodiments, SEQ ID NO: 14 is encoded by a nucleotide sequence comprising SEQ ID NO: 67. In some embodiments, SEQ ID NO: 14 is encoded by a nucleotide sequence consisting of SEQ ID NO: 67.

In some embodiments, the signaling region further comprises at least one self-assembly domain. In some embodiments, a self-assembly domain induces or initiates self-assembly. In some embodiments, self-assembly is self-assembly of monomers into multimers. In some embodiments, a multimer is a trimer. In some embodiments, the self-assembly domain is a coiled coil domain (CCD). In some embodiments, the CCD is the CCD of yeast GCN4. In some embodiments, the CCD is the CCD of a leucine zipper DNA-binding motif. In some embodiments, the motif is from yeast GCN4. In some embodiments, the GCN4 CCD comprises HMMKQIEDKIEEILSKIYHIENEIARIKKLIGEEL (SEQ ID NO: 31). In some embodiments, the GCN4 CCD consists of SEQ ID NO: 31. In some embodiments, the self-assembly domain comprises SEQ ID NO: 31. In some embodiments, the self-assembly domain consists of SEQ ID NO: 31. In some embodiments, the GCN4 CCD comprises MKQIEDKIEEILSKIYHIENEIARIKKLIGEEL (SEQ ID NO: 68). In some embodiments, the GCN4 CCD consists of SEQ ID NO: 68. In some embodiments, the self-assembly domain comprises SEQ ID NO: 68. In some embodiments, the self-assembly domain consists of SEQ ID NO: 31. In some embodiments, SEQ ID NO: 31 is encoded by a nucleotide sequence comprising SEQ ID NO: 69. In some embodiments, SEQ ID NO: 31 is encoded by a nucleotide sequence consisting of SEQ ID NO: 69. In some embodiments, SEQ ID NO: 68 is encoded by a nucleotide sequence comprising SEQ ID NO: 70. In some embodiments, SEQ ID NO: 68 is encoded by a nucleotide sequence consisting of SEQ ID NO: 70.

In some embodiments, the signaling region comprises trimeric CD40. In some embodiments, the signaling region comprises a trimeric CD40 signaling element. In some embodiments, the trimeric CD40 comprises HMMKQIEDKIEEILSKIYHIENEIARIKKLIGEELKKVAKKPTNKAPHPKQEPQEINFPDD LPGSNTAAPVQETLHGCQPVTQEDGKESRISVQERQ (SEQ ID NO: 13). In some embodiments, the trimeric CD40 consists of SEQ ID NO: 13. In some embodiments, the trimeric CD40 comprises three repeats of a CD40 monomer. In some embodiments, the element of a T cell costimulatory receptor of TNFR family protein comprises SEQ ID NO: 13. In some embodiments, the element of a T cell costimulatory receptor of TNFR family protein consists of SEQ ID NO: 13. In some embodiments, the signaling region comprises SEQ ID NO: 13. In some embodiments, the signaling region consists of SEQ ID NO: 13. In some embodiments, SEQ ID NO: 13 is encoded by a nucleotide sequence comprising SEQ ID NO: 71. In some embodiments, SEQ ID NO: 13 is encoded by a nucleotide sequence consisting of SEQ ID NO: 71. In some embodiments, the signaling region comprises a self-assembly domain and a CD40 trimer domain. In some embodiments, the signaling region comprises SEQ ID NO: 72. In some embodiments, the signaling region consists of SEQ ID NO: 72. In some embodiments, SEQ ID NO: 72 is encoded by a nucleotide sequence comprising SEQ ID NO: 73. In some embodiments, SEQ ID NO: 72 is encoded by a nucleotide sequence consists of SEQ ID NO: 73.

In some embodiments, the cytoplasmic region of the chimeric anti-inflammatory cytokines includes the complete cytoplasmic region of the T cell costimulatory receptor of the TNFR family. In some embodiments, the T cell costimulatory receptor of the TNFR family is selected from a CD40, CD27, 4-1BB (CD137), OX40 (CD134), herpesvirus entry mediator (HVEM, TNFRSF14), CD30, and glucocorticoid-induced TNFR-related protein (GITR). In some embodiments, the T cell costimulatory receptor of the TNFR family is a human T cell costimulatory receptor of the TNFR family. In some embodiments, the T cell costimulatory receptor of the TNFR family is CD40. In some embodiments, the T cell costimulatory receptor of the TNFR family is constitutively active CD40 (caCD40). In some embodiments, the T cell costimulatory receptor of the TNFR family is human CD40 or human caCD40.

The term “constitutively active CD40” or “caCD40” as used herein refers to a previously developed constitutively active CD40 (active independently of ligand binding, Levin et al., 2018), based on homo-oligomerization of CD40 signalling elements. In this case the caCD40 used included a trimeric CD40 element linked to the leucine zipper DNA-binding motif from the yeast transcription factor GCN4, which acts as a self-assembly domain.

In certain embodiments, the at least one self-assembly domain is selected from a yeast GCN4 leucine zipper DNA-binding motif and a binding site for a chemical inducer of dimerization (CID) molecule such as rapamycin or a rapamycin analog. The rapamycin analog may be Sirolimus, Everolimus, Temsirolimus, Oeforolimus, C-20-methyllyrlrapamycin (MaRap), C 16 (S)-Butylsulfonamidorapamycin (C 16-BSRap), C16-(S)-3-mehylindolerapamycin (C16-iRap), C16-(S)-7-10 methylindolerapamycin (AP21976/C16-AiRap), or a a small molecule CIO known as AP1903 (as detailed in WO2016135470, incorporated by reference as if fully include herein).

In certain embodiments, the yeast GCN4 leucine zipper DNA-binding motif is a motif forming homodimers, a motif forming homotrimers or a motif forming homotetramers. In some embodiments, the yeast GCN4 leucine zipper DNA-binding motif is a motif forming homotrimers.

In addition, there are quite a few natural or artificial elements which may be used for engineering homodimeric, homotrimeric or other homo-oligomeric protein configurations for use as the self-assembly domain in the present invention. For example, the receptor-binding domain (RBD) of the coronavirus spike protein (encoded by the earlier Pfizer vaccine BNT162b1) is modified by the addition of a T4 fibritin-derived foldon trimerization domain (Mulligan et al., 2020, “Phase I/II study of COVID-19 RNA vaccine BNT162b1 in adults”, Nature volume 586, pages589-593, herein incorporated by reference in its entirety).

In some embodiments, the at least one self-assembly domain is selected from a coiled-coil domain such as a coiled coil domain from a yeast GCN4 leucine zipper DNA-binding motif, e.g., a binding motif forming homodimers, a binding motif forming homotetramers, or preferably a binding motif forming homotrimers; and a binding site for a chemical inducer of dimerization (CID) molecule, such as AP1903.

In some embodiments, the at least one self-assembly domain is a yeast GCN4 leucine zipper DNA-binding motif forming homotrimers. In some embodiments, the CD40 comprises a yeast GCN4 self-assembly domain linked to a trimeric CD40 element.

In some embodiments, the cytoplasmic region of the chimeric anti-inflammatory cytokines includes the complete cytoplasmic region of TLR4. In some embodiments, the cytoplasmic region of the chimeric anti-inflammatory cytokines includes the complete cytoplasmic region of human TLR4.

In some embodiments, the regions are linked. In some embodiments, the regions are present in a single polypeptide chain. In some embodiments, at least two regions are linked by linkers. In some embodiments, all regions are linked by linkers. In some embodiments, the cytokine region and transmembrane region are linked by a linker. In some embodiments, the cytokine region and the membrane proximal region are linked by a linker. In some embodiments, the membrane proximal region and the transmembrane region are linked by a linker. In some embodiments, the transmembrane region and the signaling region are linked by a linker. The term “linked” as used herein with reference to elements which are comprised within the chimeric anti-inflammatory cytokines indicates that these elements are physically linked, i.e., are part of the same polypeptide. However, the linked elements may not necessarily be directly linked, but there may be a linker sequence or an additional element between them.

Flexible peptide linkers are well-known in the art. Empirical linkers designed by researchers are generally classified into three categories according to their structures: flexible linkers, rigid linkers, and in vivo cleavable linkers, each one of which is incorporated by reference as if fully disclosed herein.

In principle, to provide flexibility, the linkers are generally composed of small, non-polar (e.g. Gly) or polar (e.g. Ser or Thr) amino acids, such an underlying sequence of alternating Gly and Ser residues. Solubility of the linker and associated polypeptide may be enhanced by including charged residues; e.g., two positively charged residues (Lys) and one negatively charged residue (Glu). The linker may vary from 2 to 31 amino acids, optimized for each condition so that the linker does not impose any constraints on the conformation or interactions of the linked partners in lengths, such as between 12 and 18 residues.

In some embodiments, the polypeptide is a polypeptide provided in Table 1. In some embodiments, the polypeptide comprises an amino acid sequence selected from SEQ ID NO: 15-30. In some embodiments, the polypeptide consists of an amino acid sequence selected from SEQ ID NO: 15-30. In some embodiments, the polypeptide comprises an amino acid sequence selected from SEQ ID NO: 15-22. In some embodiments, the polypeptide consists of an amino acid sequence selected from SEQ ID NO: 15-22. In some embodiments, the polypeptide comprises an amino acid sequence selected from SEQ ID NO: 23-30. In some embodiments, the polypeptide consists of an amino acid sequence selected from SEQ ID NO: 23-30. In some embodiments, the polypeptide comprises SEQ ID NO: 15. In some embodiments, the polypeptide consists of SEQ ID NO: 15. In some embodiments, the polypeptide comprises SEQ ID NO: 16. In some embodiments, the polypeptide consists of SEQ ID NO: 16. In some embodiments, the polypeptide comprises SEQ ID NO: 17. In some embodiments, the polypeptide consists of SEQ ID NO: 17. In some embodiments, the polypeptide comprises SEQ ID NO: 18. In some embodiments, the polypeptide consists of SEQ ID NO: 18. In some embodiments, the polypeptide comprises SEQ ID NO: 19. In some embodiments, the polypeptide consists of SEQ ID NO: 19. In some embodiments, the polypeptide comprises SEQ ID NO: 20. In some embodiments, the polypeptide consists of SEQ ID NO: 20. In some embodiments, the polypeptide comprises SEQ ID NO: 21. In some embodiments, the polypeptide consists of SEQ ID NO: 21. In some embodiments, the polypeptide comprises SEQ ID NO: 22. In some embodiments, the polypeptide consists of SEQ ID NO: 22. In some embodiments, the polypeptide comprises SEQ ID NO: 23. In some embodiments, the polypeptide consists of SEQ ID NO: 23. In some embodiments, the polypeptide comprises SEQ ID NO: 24. In some embodiments, the polypeptide consists of SEQ ID NO: 24. In some embodiments, the polypeptide comprises SEQ ID NO: 25. In some embodiments, the polypeptide consists of SEQ ID NO: 25. In some embodiments, the polypeptide comprises SEQ ID NO: 26. In some embodiments, the polypeptide consists of SEQ ID NO: 26. In some embodiments, the polypeptide comprises SEQ ID NO: 27. In some embodiments, the polypeptide consists of SEQ ID NO: 27. In some embodiments, the polypeptide comprises SEQ ID NO: 28. In some embodiments, the polypeptide consists of SEQ ID NO: 28. In some embodiments, the polypeptide comprises SEQ ID NO: 29. In some embodiments, the polypeptide consists of SEQ ID NO: 29. In some embodiments, the polypeptide comprises SEQ ID NO: 30. In some embodiments, the polypeptide consists of SEQ ID NO: 30. In some embodiments, the polypeptide comprises a sequence selected from SEQ ID NO: 19-22. In some embodiments, the polypeptide consists of a sequence selected from SEQ ID NO: 19-22. In some embodiments, the polypeptide comprises a sequence selected from SEQ ID NO: 27-30. In some embodiments, the polypeptide consists of a sequence selected from SEQ ID NO: 27-30. In some embodiments, the nucleic acid molecule comprises a nucleotide sequence selected from SEQ ID NO: 32-47. In some embodiments, the nucleic acid molecule consists of a nucleotide sequence selected from SEQ ID NO: 32-47. In some embodiments, the nucleic acid molecule comprises a nucleotide sequence selected from SEQ ID NO: 36-39. In some embodiments, the nucleic acid molecule consists of a nucleotide sequence selected from SEQ ID NO: 36-39. In some embodiments, the nucleic acid molecule comprises a nucleotide sequence selected from SEQ ID NO: 44-47. In some embodiments, the nucleic acid molecule consists of a nucleotide sequence selected from SEQ ID NO: 44-47.

In some embodiments, molecule 2101 is scIL-10+CFR-bridge+CD40TM+CD40-IR (mono). In some embodiments, molecule 2101 is MHSSALLCCLVLLTGVRASPGQGTQSENSCTHFPGNLPNMLRDLRDAFSRVKTFFQMK DQLDNLLLKESLLEDFKGYLGCQALSEMIQFYLEEVMPQAENQDPDIKAHVNSLGENL KTLRLRLRRCHRFLPCENKSKAVEQVKNAFNKLQEKGIYKAMSEFDIFINYIEAYMTM KIRNGSTSGSGKPGSGEGSTKGSPGQGTQSENSCTHFPGNLPNMLRDLRDAFSRVKTFF QMKDQLDNLLLKESLLEDFKGYLGCQALSEMIQFYLEEVMPQAENQDPDIKAHVNSL GENLKTLRLRLRRCHRFLPCENKSKA VEQVKNAFNKLQEKGIYKAMSEFDIFINYIEAY MTMKIRNPGGGGGSGGGSGGGSQLPVLSLNITCQMNKLEALVVIPIIFGILFAILLVLVFI ELKKVAKKPTNKAPHPKQEPQEINFPDDLPGSNTAAPVQETLHGCQPVTQEDGKESRIS VQERQ (SEQ ID NO: 15). In some embodiments, molecule 2101 lacks the signal peptide and is SEQ ID NO: 23. In some embodiments, SEQ ID NO: 15 is encoded by a nucleotide sequence comprising SEQ ID NO: 32. In some embodiments, SEQ ID NO: 15 is encoded by a nucleotide sequence consisting of SEQ ID NO: 32. In some embodiments, SEQ ID NO: 23 is encoded by a nucleotide sequence comprising SEQ ID NO: 40. In some embodiments, SEQ ID NO: 23 is encoded by a nucleotide sequence consisting of SEQ ID NO: 40.

In some embodiments, molecule 2102 is scIL-10+CFR-bridge+CD40TM+GCN4-CD40. In some embodiments, molecule 2102 is MHSSALLCCLVLLTGVRASPGQGTQSENSCTHFPGNLPNMLRDLRDAFSRVKTFFQMK DQLDNLLLKESLLEDFKGYLGCQALSEMIQFYLEEVMPQAENQDPDIKAHVNSLGENL KTLRLRLRRCHRFLPCENKSKAVEQVKNAFNKLQEKGIYKAMSEFDIFINYIEAYMTM KIRNGSTSGSGKPGSGEGSTKGSPGQGTQSENSCTHFPGNLPNMLRDLRDAFSRVKTFF QMKDQLDNLLLKESLLEDFKGYLGCQALSEMIQFYLEEVMPQAENQDPDIKAHVNSL GENLKTLRLRLRRCHRFLPCENKSKAVEQVKNAFNKLQEKGIYKAMSEFDIFINYIEAY MTMKIRNPGGGGGSGGGSGGGSQLPVLSLNITCQMNKLEALVVIPIIFGILFAILLVLVFI HMMKQIEDKIEEILSKIYHIENEIARIKKLIGEELKKVAKKPTNKAPHPKQEPQEINFPDD LPGSNTAAPVQETLHGCQPVTQEDGKESRISVQERQ (SEQ ID NO: 16). In some embodiments, molecule 2102 lacks the signal peptide and is SEQ ID NO: 24. In some embodiments, SEQ ID NO: 16 is encoded by a nucleotide sequence comprising SEQ ID NO: 33. In some embodiments, SEQ ID NO: 16 is encoded by a nucleotide sequence consisting of SEQ ID NO: 33. In some embodiments, SEQ ID NO: 24 is encoded by a nucleotide sequence comprising SEQ ID NO: 41. In some embodiments, SEQ ID NO: 24 is encoded by a nucleotide sequence consisting of SEQ ID NO: 41.

In some embodiments, molecule 2103 is scIL-10+CFR-bridge+HLA-TM+CD40-IR (mono). In some embodiments, molecule 2103 is MHSSALLCCLVLLTGVRASPGQGTQSENSCTHFPGNLPNMLRDLRDAFSRVKTFFQMK DQLDNLLLKESLLEDFKGYLGCQALSEMIQFYLEEVMPQAENQDPDIKAHVNSLGENL KTLRLRLRRCHRFLPCENKSKA VEQVKNAFNKLQEKGIYKAMSEFDIFINYIEAYMTM KIRNGSTSGSGKPGSGEGSTKGSPGQGTQSENSCTHFPGNLPNMLRDLRDAFSRVKTFF QMKDQLDNLLLKESLLEDFKGYLGCQALSEMIQFYLEEVMPQAENQDPDIKAHVNSL GENLKTLRLRLRRCHRFLPCENKSKAVEQVKNAFNKLQEKGIYKAMSEFDIFINYIEAY MTMKIRNPGGGGGSGGGSGGGSQLPVLSLNITCQMNKLEVGIIAGLVLFGAVITGAVV AAVMWELKKVAKKPTNKAPHPKQEPQEINFPDDLPGSNTAAPVQETLHGCQPVTQED GKESRISVQERQ (SEQ ID NO: 17). In some embodiments, molecule 2103 lacks the signal peptide and is SEQ ID NO: 25. In some embodiments, SEQ ID NO: 17 is encoded by a nucleotide sequence comprising SEQ ID NO: 34. In some embodiments, SEQ ID NO: 17 is encoded by a nucleotide sequence consisting of SEQ ID NO: 34. In some embodiments, SEQ ID NO: 25 is encoded by a nucleotide sequence comprising SEQ ID NO: 42. In some embodiments, SEQ ID NO: 25 is encoded by a nucleotide sequence consisting of SEQ ID NO: 42.

In some embodiments, molecule 2104 is scIL-10+CFR-bridge+HLA-TM+GCN4-CD40. In some embodiments, molecule 2104 is MHSSALLCCLVLLTGVRASPGQGTQSENSCTHFPGNLPNMLRDLRDAFSRVKTFFQMK DQLDNLLLKESLLEDFKGYLGCQALSEMIQFYLEEVMPQAENQDPDIKAHVNSLGENL KTLRLRLRRCHRFLPCENKSKAVEQVKNAFNKLQEKGIYKAMSEFDIFINYIEAYMTM KIRNGSTSGSGKPGSGEGSTKGSPGQGTQSENSCTHFPGNLPNMLRDLRDAFSRVKTFF QMKDQLDNLLLKESLLEDFKGYLGCQALSEMIQFYLEEVMPQAENQDPDIKAHVNSL GENLKTLRLRLRRCHRFLPCENKSKAVEQVKNAFNKLQEKGIYKAMSEFDIFINYIEAY MTMKIRNPGGGGGSGGGSGGGSQLPVLSLNITCQMNKLEVGIIAGLVLFGAVITGAVV AAVMWHMMKQIEDKIEEILSKIYHIENEIARIKKLIGEELKKVAKKPTNKAPHPKQEPQ EINFPDDLPGSNTAAPVQETLHGCQPVTQEDGKESRISVQERQ (SEQ ID NO: 18). In some embodiments, molecule 2104 lacks the signal peptide and is SEQ ID NO: 26. In some embodiments, SEQ ID NO: 18 is encoded by a nucleotide sequence comprising SEQ ID NO: 35. In some embodiments, SEQ ID NO: 18 is encoded by a nucleotide sequence consisting of SEQ ID NO: 35. In some embodiments, SEQ ID NO: 26 is encoded by a nucleotide sequence comprising SEQ ID NO: 43. In some embodiments, SEQ ID NO: 26 is encoded by a nucleotide sequence consisting of SEQ ID NO: 43.

In some embodiments, molecule 2105 is scIL-10+HLA-A2-bridge+CD40TM+CD40-IR (mono). In some embodiments, molecule 2105 is MHSSALLCCLVLLTGVRASPGQGTQSENSCTHFPGNLPNMLRDLRDAFSRVKTFFQMK DQLDNLLLKESLLEDFKGYLGCQALSEMIQFYLEEVMPQAENQDPDIKAHVNSLGENL KTLRLRLRRCHRFLPCENKSKAVEQVKNAFNKLQEKGIYKAMSEFDIFINYIEAYMTM KIRNGSTSGSGKPGSGEGSTKGSPGQGTQSENSCTHFPGNLPNMLRDLRDAFSRVKTFF QMKDQLDNLLLKESLLEDFKGYLGCQALSEMIQFYLEEVMPQAENQDPDIKAHVNSL GENLKTLRLRLRRCHRFLPCENKSKAVEQVKNAFNKLQEKGIYKAMSEFDIFINYIEAY MTMKIRNPGGGGGSGGGSGGGSQLLRWEPSSQPTIPILEALVVIPIIFGILFAILLVLVFIE LKKVAKKPTNKAPHPKQEPQEINFPDDLPGSNTAAPVQETLHGCQPVTQEDGKESRISV QERQ (SEQ ID NO: 19). In some embodiments, molecule 2105 lacks the signal peptide and is SEQ ID NO: 27. In some embodiments, SEQ ID NO: 19 is encoded by a nucleotide sequence comprising SEQ ID NO: 36. In some embodiments, SEQ ID NO: 19 is encoded by a nucleotide sequence consisting of SEQ ID NO: 36. In some embodiments, SEQ ID NO: 27 is encoded by a nucleotide sequence comprising SEQ ID NO: 44. In some embodiments, SEQ ID NO: 27 is encoded by a nucleotide sequence consisting of SEQ ID NO: 44.

In some embodiments, molecule 2106 is scIL-10+HLA-A2-bridge+CD40TM+GCN4-CD40. In some embodiments, molecule 2106 is MHSSALLCCLVLLTGVRASPGQGTQSENSCTHFPGNLPNMLRDLRDAFSRVKTFFQMK DQLDNLLLKESLLEDFKGYLGCQALSEMIQFYLEEVMPQAENQDPDIKAHVNSLGENL KTLRLRLRRCHRFLPCENKSKAVEQVKNAFNKLQEKGIYKAMSEFDIFINYIEAYMTM KIRNGSTSGSGKPGSGEGSTKGSPGQGTQSENSCTHFPGNLPNMLRDLRDAFSRVKTFF QMKDQLDNLLLKESLLEDFKGYLGCQALSEMIQFYLEEVMPQAENQDPDIKAHVNSL GENLKTLRLRLRRCHRFLPCENKSKAVEQVKNAFNKLQEKGIYKAMSEFDIFINYIEAY MTMKIRNPGGGGGSGGGSGGGSQLLRWEPSSQPTIPILEALVVIPIIFGILFAILLVLVFIH MMKQIEDKIEEILSKIYHIENEIARIKKLIGEELKKVAKKPTNKAPHPKQEPQEINFPDDL PGSNTAAPVQETLHGCQPVTQEDGKESRISVQERQ (SEQ ID NO: 20). In some embodiments, molecule 2106 lacks the signal peptide and is SEQ ID NO: 28. In some embodiments, SEQ ID NO: 20 is encoded by a nucleotide sequence comprising SEQ ID NO: 37. In some embodiments, SEQ ID NO: 20 is encoded by a nucleotide sequence consisting of SEQ ID NO: 37. In some embodiments, SEQ ID NO: 28 is encoded by a nucleotide sequence comprising SEQ ID NO: 45. In some embodiments, SEQ ID NO: 28 is encoded by a nucleotide sequence consisting of SEQ ID NO: 45.

In some embodiments, molecule 2107 is scIL-10+HLA-A2-bridge+HLA-TM+CD40-IR (mono). In some embodiments, molecule 2107 is MHSSALLCCLVLLTGVRASPGQGTQSENSCTHFPGNLPNMLRDLRDAFSRVKTFFQMK DQLDNLLLKESLLEDFKGYLGCQALSEMIQFYLEEVMPQAENQDPDIKAHVNSLGENL KTLRLRLRRCHRFLPCENKSKAVEQVKNAFNKLQEKGIYKAMSEFDIFINYIEAYMTM KIRNGSTSGSGKPGSGEGSTKGSPGQGTQSENSCTHFPGNLPNMLRDLRDAFSRVKTFF QMKDQLDNLLLKESLLEDFKGYLGCQALSEMIQFYLEEVMPQAENQDPDIKAHVNSL GENLKTLRLRLRRCHRFLPCENKSKAVEQVKNAFNKLQEKGIYKAMSEFDIFINYIEAY MTMKIRNPGGGGGSGGGSGGGSQLLRWEPSSQPTIPILEVGIIAGLVLFGAVITGAVVA AVMWELKKVAKKPTNKAPHPKQEPQEINFPDDLPGSNTAAPVQETLHGCQPVTQEDG KESRISVQERQ (SEQ ID NO: 21). In some embodiments, molecule 2107 lacks the signal peptide and is SEQ ID NO: 29. In some embodiments, SEQ ID NO: 21 is encoded by a nucleotide sequence comprising SEQ ID NO: 38. In some embodiments, SEQ ID NO: 21 is encoded by a nucleotide sequence consisting of SEQ ID NO: 38. In some embodiments, SEQ ID NO: 29 is encoded by a nucleotide sequence comprising SEQ ID NO: 46. In some embodiments, SEQ ID NO: 29 is encoded by a nucleotide sequence consisting of SEQ ID NO: 46.

In some embodiments, molecule 2108 is scIL-10+HLA-A2-bridge+HLA-TM+GCN4-CD40. In some embodiments, molecule 2108 is MHSSALLCCLVLLTGVRASPGQGTQSENSCTHFPGNLPNMLRDLRDAFSRVKTFFQMK DQLDNLLLKESLLEDFKGYLGCQALSEMIQFYLEEVMPQAENQDPDIKAHVNSLGENL KTLRLRLRRCHRFLPCENKSKAVEQVKNAFNKLQEKGIYKAMSEFDIFINYIEAYMTM KIRNGSTSGSGKPGSGEGSTKGSPGQGTQSENSCTHFPGNLPNMLRDLRDAFSRVKTFF QMKDQLDNLLLKESLLEDFKGYLGCQALSEMIQFYLEEVMPQAENQDPDIKAHVNSL GENLKTLRLRLRRCHRFLPCENKSKAVEQVKNAFNKLQEKGIYKAMSEFDIFINYIEAY MTMKIRNPGGGGGSGGGSGGGSQLLRWEPSSQPTIPILEVGIIAGLVLFGAVITGAVVA AVMWHMMKQIEDKIEEILSKIYHIENEIARIKKLIGEELKKVAKKPTNKAPHPKQEPQEI NFPDDLPGSNTAAPVQETLHGCQPVTQEDGKESRISVQERQ (SEQ ID NO: 22). In some embodiments, molecule 2108 lacks the signal peptide and is SEQ ID NO: 30. In some embodiments, SEQ ID NO: 22 is encoded by a nucleotide sequence comprising SEQ ID NO: 39. In some embodiments, SEQ ID NO: 22 is encoded by a nucleotide sequence consisting of SEQ ID NO: 39. In some embodiments, SEQ ID NO: 30 is encoded by a nucleotide sequence comprising SEQ ID NO: 47. In some embodiments, SEQ ID NO: 30 is encoded by a nucleotide sequence consisting of SEQ ID NO: 47.

In some embodiments, the transmembrane domain and signaling domain comprise LEALVVIPIIFGILFAILLVLVFIELKKVAKKPTNKAPHPKQEPQEINFPDDLPGSNTAAPV QETLHGCQPVTQEDGKESRISVQERQ (SEQ ID NO: 74) n some embodiments, the transmembrane domain and signaling domain comprise consists of SEQ ID NO: 74 In some embodiments, SEQ ID NO: 74 is encoded by a nucleotide sequence comprising SEQ ID NO: 75. In some embodiments, SEQ ID NO: 74 is encoded by a nucleotide sequence consists of SEQ ID NO: 75.

In some embodiments, the transmembrane domain and signaling domain comprise LEALVVIPIIFGILFAILLVLVFIHMMKQIEDKIEEILSKIYHIENEIARIKKLIGEELKKVAK KPTNKAPHPKQEPQEINFPDDLPGSNTAAPVQETLHGCQPVTQEDGKESRISVQERQ (SEQ ID NO: 76) n some embodiments, the transmembrane domain and signaling domain comprise consists of SEQ ID NO: 76 In some embodiments, SEQ ID NO: 76 is encoded by a nucleotide sequence comprising SEQ ID NO: 77. In some embodiments, SEQ ID NO: 76 is encoded by a nucleotide sequence consists of SEQ ID NO: 77.

In some embodiments, the transmembrane domain and signaling domain comprise LEALVVIPIIFGILFAILLVLVFIHMMKQIEDKIEEILSKIYHIENEIARIKKLIGEELKKVAK KPTNKAPHPKQEPQEINFPDDLPGSNTAAPVQETLHGCQPVTQEDGKESRISVQERQ (SEQ ID NO: 78) n some embodiments, the transmembrane domain and signaling domain comprise consists of SEQ ID NO: 78 In some embodiments, SEQ ID NO: 78 is encoded by a nucleotide sequence comprising SEQ ID NO: 79. In some embodiments, SEQ ID NO: 78 is encoded by a nucleotide sequence consists of SEQ ID NO: 79.

In some embodiments, the transmembrane domain and signaling domain comprise LEVGIIAGLVLFGA VITGAVVAAVMWHMMKQIEDKIEEILSKIYHIENEIARIKKLIGEE LKKVAKKPTNKAPHPKQEPQEINFPDDLPGSNTAAPVQETLHGCQPVTQEDGKESRISV QERQ (SEQ ID NO: 80) n some embodiments, the transmembrane domain and signaling domain comprise consists of SEQ ID NO: 80 In some embodiments, SEQ ID NO: 80 is encoded by a nucleotide sequence comprising SEQ ID NO: 81. In some embodiments, SEQ ID NO: 80 is encoded by a nucleotide sequence consists of SEQ ID NO: 81.

The inventors have succeeded in creating constitutively active, ligand-independent CD40 molecule, which confers a constitutively activated state on transfected T cells, by linking CD40 to an assembly domain from the GCN4 yeast transcriptional activator, which contains a leucine zipper DNA-binding motif that induces homophilic interactions, and thus induces self-oligomerizing. Since T cell costimulatory receptors of the TNFR family mentioned below are structurally similar and all signal through adaptor TNFR-associated factor (TRAF) proteins via the NF-KB, p38 MAPK or JNK/SAPK pathways, one may assume that other TNFR members, when linked to a self-assembly domain as described for CD40, will similarly become constitutively active and capable of conferring a constitutively activated state on transfected T cells.

In certain embodiments, any of the elements comprising the chimeric anti-inflammatory cytokine, especially the cytokine, the signalling element of a T cell costimulatory receptor of the tumor necrosis factor receptor (TNFR) family, and the toll/interleukin-1 receptor (TIR) signaling element in any one of the above defined embodiments are derived from a human sequence. In some embodiments, the proteins recited herein are human proteins unless specified otherwise.

By another aspect, there is provided a polypeptide encoded by a nucleic acid molecule of the invention. In a further aspect, the present invention provides a polypeptide encoded by the nucleic acid of any of the above embodiments.

In a further aspect, the present invention provides a polypeptide comprising a chimeric anti-inflammatory cytokine including: at least one single-chain anti-inflammatory cytokine linked to a transmembrane region which is linked to a cytoplasmic region including at least one signalling element of a T cell costimulatory receptor of the tumor necrosis factor receptor (TNFR) family optionally linked to at least one self-assembly domain. This polypeptide may be encoded by any of the nucleic acids of the invention as described above. Alternatively, this polypeptide of the invention may be encoded by more than a single nucleic acid.

By another aspect, there is provided a cell comprising a nucleic acid molecule of the invention. By another aspect, there is provided a cell comprising a vector of the invention. By another aspect, there is provided a cell comprising a polypeptide of the invention.

In a further aspect, the present invention provides an immune cell comprising the nucleic acid molecule of any one of above embodiments, or the vector of any one of above embodiments. In some embodiments, the immune cell is a CD4+ T cell. It is noted that the presence of the nucleic acid molecule of the invention in the immune cell causes the immune cell to become a regulatory immune cell. For example, the CD4+ T cells becomes a regulatory T cell following introduction of the nucleic acid of the invention. When the above nucleic acid molecule is expressed in a T cell it acts in cis when the cytokine receptor is also expressed in the same T cell.

In some embodiments, the cell is a target cell. In some embodiments, the cell is an engineered cell. In some embodiments, the cell is an immune cell. In some embodiments, the immune cell is a lymphocyte. In some embodiments, the immune cell is a T cell. In some embodiments, the T cell is a CD4 T cell. In some embodiments, the T cell is a T helper cell. In some embodiments, the T cell becomes a Treg due to the presence of the nucleic acid molecule, vector or polypeptide. In some embodiments, the cells are peripheral blood mononuclear cells (PBMCs). In some embodiments, the immune cells are peripheral blood immune cells. In certain embodiments, the immune cells are autologous peripheral immune cells, e.g., autologous CD4+ T cells.

In some embodiments, the cell comprises increased section of at least one cytokine. In some embodiments, the cytokine is an anti-inflammatory cytokine. In some embodiments, the cytokine is IL-10. In some embodiments, the cytokine is interferon gamma (IFNg). In some embodiments, increased is as compared to a control cell. In some embodiments, a control cell is a cell that does not comprise the nucleic acid molecule, vector or polypeptide of the invention. In some embodiments, a control cell is a non-engineered cell. In some embodiments, a control cell is a primary cell. In some embodiments, a control cell is a cell in culture. In some embodiments, a control cell is an expanded cell. In some embodiments, the control cell is the same cell type as the target cell. In some embodiments, the control cell is the same cell type as the target cell before it expressed the nucleic acid molecule, vector or polypeptide of the invention.

In some embodiments, increased comprises at least a 5, 10, 15, 20, 25, 30, 35, 40, 45, 50, 55, 60, 65, 70, 75, 80, 85, 90, 92, 95, 97, 99 or 100% increase. Each possibility represents a separate embodiment of the invention. In some embodiments, increased comprises at least a 50% increase. In some embodiments, increased comprises at least a 100% increase. In some embodiments, increased is significantly increased. In some embodiments, significant is statistically significant.

By another aspect, there is provided a population of cells, wherein at least 10% of the cells in the population comprises a nucleic acid molecule of the invention, a vector of the invention or a polypeptide of the invention.

In some embodiments, the population is an isolated population. In some embodiments, the population is an enriched population. In some embodiments, the population is a purified population. In some embodiments, the population is an in vitro population. In some embodiments, the population is in culture. In some embodiments, the population is an expanded population. In one embodiment, T cells are expanded using methods known in the art. Expanded T cells that express the molecule of the invention may be administered back to a subject. In another embodiment PBMCs are transduced or transfected with nucleic acid molecules of the invention and administered to a subject. In some embodiments, T cells expressing a nucleic acid molecule, vector or polypeptide of the invention are expanded and administered back to a subject.

In some embodiments, at least 10% is at least 20, 25, 30, 35, 40, 45, 50, 55, 60, 65, 70, 75, 80, 85, 90, 92, 95, 97, 99 or 100%. Each possibility represents a separate embodiment of the invention. In some embodiments, at least 10% is at least 20%. In some embodiments, at least 10% is at least 50%.

In some embodiments, the cells are from a subject. In some embodiments, the cells are autologous to the subject. In some embodiments, the cells are allogenic to the subject. In some embodiments, the cells are syngeneic to the subject. In some embodiments, the cells are extracted from the subject. In some embodiments, the cells are expanded after extraction from the subject.

In still another aspect, the present invention provides a method of preparing engineered regulatory immune cells comprising the steps of: (a) obtaining immune cells and expanding them; and (b) transducing the immune cells with the nucleic acids or the vector defined above, thereby obtaining engineered regulatory immune cells.

In a further aspect, the present invention provides an engineered regulatory immune cell obtained by any one of the methods defined above.

By another aspect, there is provided a method of converting a CD4 positive T cell is a regulatory T cell (Treg), the method comprising introducing into the CD4 positive T cell a nucleic acid molecule of the invention, a vector of the invention or a polypeptide of the invention, thereby converting a CD4 positive T cell into a Treg.

In some embodiments, the CD4 positive T cell is not a Treg. In some embodiments, the CD4 positive T cell is a primary cell. In some embodiments, the primary cell is isolated from peripheral blood. In some embodiments, the method further comprises obtaining the CD4 positive T cell. In some embodiments, the method further comprises receiving the CD4 positive T cell. In some embodiments, the T cell is from a subject. In some embodiments, the method is an ex vivo method. In some embodiments, the method is an in vitro method. In some embodiments, the method is performed in culture. In some embodiments, the introducing comprises transfection. In some embodiments, the introducing comprises infection. In some embodiments, infection comprises viral, lentiviral or adenoviral transduction. In some embodiments, introducing comprises genome editing.

By another aspect, there is provided a Treg produced by a method of the invention. In some embodiments, the Treg is an artificial Treg. In some embodiments, the Treg is a synthetic Treg. In some embodiments, the Treg is an engineered Treg.

By another aspect, there is provided a composition comprising a nucleic acid molecule of the invention, a vector of the invention, a polypeptide of the invention, a cell of the invention, a population of the invention or a Treg of the invention.

In some embodiments, the composition is pharmaceutical composition. In some embodiments, the composition is a therapeutic composition. In some embodiments, the composition is a diagnostic composition.

In some embodiments, the composition comprises a pharmaceutically acceptable carrier, excipient or adjuvant. As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the composition comprises a therapeutically effective amount of the nucleic acid molecule of the invention. In some embodiments, the composition comprises a therapeutically effective amount of the vector of the invention. In some embodiments, the composition comprises a therapeutically effective amount of the polypeptide of the invention. In some embodiments, the composition comprises a therapeutically effective amount of the cell of the invention. In some embodiments, the composition comprises a therapeutically effective amount of the Treg of the invention. In some embodiments, the composition comprises a therapeutically effective amount of the population of the invention. In some embodiments, the population comprises at least 1 million cells. As used herein, the term “therapeutically effective amount” refers to an amount of an active agent to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

In some embodiments, the composition is formulated to systemic administration. In some embodiments, the composition is formulated for administration to a subject. In some embodiments, the subject is a human. It will be understood by a skilled artisan that cells in a standard culture, which contains media from animals (e.g., FBS) are not suitable for administration to a human. Chemically defined media can be used, or the cells can be isolated and washed and moved to an acceptable carrier medium. In some embodiments, the composition is formulated for intravenous administration.

By another aspect, there is provided a method for inducing immune suppression in a subject, the method comprising administering to the subject a composition of the invention, thereby inducing immune suppression.

In some embodiments, the subject is a mammal. In some embodiments, the mammal is a human. In some embodiments, the subject is in need of a method of the invention. In some embodiments, the subject suffers from a disease, condition or disorder treatable by immune suppression. In some embodiments, the subject suffers from a disease, condition or disorder characterized by excessive activity of the immune system. In some embodiments, excessive activity is overactivity. In some embodiments, excessive activity is aberrant activity. In some embodiments, the subject suffers from an autoimmune disease. In some embodiments, the subject suffers from an inflammatory disease. In some embodiments, the subject is treatable by the administration of Tregs.

By another aspect, there is provided a method of treating a disease, disorder or condition in a subject, the method comprising administering to the subject a composition of the invention, thereby treating a disease, disorder or condition.

In yet an additional aspect, the present invention provides the nucleic acid molecule of any one of above embodiments, the vector of any one of above embodiments, or the immune cell of any one of above embodiments, for use in treating a disease, disorder or condition manifested in excessive activity of the immune system, such as an autoimmune or inflammatory disease or disorder or condition. In some embodiments, the nucleic acid molecule is for use in a method of the invention. In some embodiments, the vector is for use in a method of the invention. In some embodiments, the polypeptide is for use in a method of the invention. In some embodiments, the cell is for use in a method of the invention. In some embodiments, the population is for use in a method of the invention. In some embodiments, the Treg is for use in a method of the invention.

In yet an additional aspect, the present invention provides a method for treating a disease, disorder or condition manifested in excessive activity of the immune system, such as an autoimmune or inflammatory disease or disorder or condition, comprising administering to a subject in need thereof the nucleic acid molecule of any one of above embodiments, the vector of any one of above embodiments, or the immune cell of any one of above embodiments.

In certain embodiments, the disease, disorder or condition manifested in excessive activity of the immune system is selected from an autoimmune disease, allergy, asthma, and response to an organ or bone marrow transplantation. In certain embodiments, the autoimmune disease is selected from type 1 diabetes; rheumatoid arthritis; psoriasis; psoriatic arthritis; multiple sclerosis; systemic lupus erythematosus; inflammatory bowel disease, such as Crohn's disease and ulcerative colitis; Addison's disease; Graves' disease; Sjögren's syndrome; Hashimoto's thyroiditis; myasthenia gravis; vasculitis; pernicious anemia; celiac disease; and atherosclerosis.

In some embodiments, the disease, disorder of condition is an autoimmune disease, disorder or condition. In some embodiments, the disease, disorder of condition is an inflammatory disease, disorder or condition. In some embodiments, the disease, disorder of condition is selected from transplant rejection, cardiovascular disease, obesity, systemic inflammation, celiac disease, dermatomyositis, Graves' disease, Addison's disease, Hashimoto disease, Multiple sclerosis, Crohn's disease, colitis, myasthenia gravis, pernicious anemia, arthritis, Sjogren syndrome, lupus, diabetes, pemphigus vulgaris, pemphigus follicularis, membranous nephropathy, sarcoidosis, vasculitis, atherosclerosis, granulomatosis, spondyloarthropathy and cytokine storm. In some embodiments, transplant rejection is potential transplant rejection. In some embodiments, the subject has received a transplant. In some embodiments, the transplant is an organ transplant. In some embodiments, the transplant is a bone marrow transplant. In some embodiments, the disease is graft versus host disease. In some embodiments, the disease or condition is inflammatory bowel disease (IBD). In some embodiments, IBD comprises Crohn's disease and colitis. In some embodiments, colitis is ulcerative colitis. In some embodiments, diabetes is type 1 diabetes. In some embodiments, diabetes is type 2 diabetes. In some embodiments, Hashimoto's is Hashimoto's thyroiditis. In some embodiments, lupus is lupus erythematosus. In some embodiments, lupus is systemic lupus erythematosus. It will be understood by a skilled artisan that the disease, disorder or condition is any one that can be treated by reducing the activity of the immune system and specifically by the introduction of Tregs.

Additionally, the present invention includes the following embodiments:
Embodiment 1: A nucleic acid molecule comprising a nucleotide sequence encoding a polypeptide comprising a chimeric anti-inflammatory cytokine including: at least one single-chain anti-inflammatory cytokine linked to a transmembrane region which is linked to a cytoplasmic region including at least one signalling element of a T cell costimulatory receptor of the tumor necrosis factor receptor (TNFR) family optionally linked to at least one self-assembly domain.
Embodiment 2: The nucleic acid molecule of Embodiment 1, wherein the cytoplasmic region further includes at least one toll/interleukin-1 receptor (TIR) signalling element, such as a TIR signalling element from a toll-like receptor such as TLR4, an Interleukin-1 receptor, or a cytosolic adaptor protein such as MyD88.
Embodiment 3: The nucleic acid molecule of Embodiment 1 or 2, wherein the at least one single-chain cytokine is selected from a single-chain IL-10.
Embodiment 4: The nucleic acid molecule of any one of Embodiments 1 to 3, wherein the transmembrane region is selected from a transmembrane region of the heavy chain of a human MHC class I molecule selected from an HLA-A, HLA-B and HLA-C, preferably HLA-A2; a transmembrane region of CD40; a transmembrane region of TLR4; and a transmembrane region of human CD28.
Embodiment 5: The nucleic acid molecule of any one of Embodiments 1 to 4, wherein the T cell costimulatory receptor of the TNFR family is selected from a CD40, CD27, 4-1BB (CD137), OX40 (CD134), herpesvirus entry mediator (HVEM, TNFRSF14), CD30, and glucocorticoid-induced TNFR-related protein (GITR).
Embodiment 6: The nucleic acid molecule of any one of Embodiments 1 to 5, wherein the at least one self-assembly domain is selected from a coiled-coil domain such as a coiled coil domain from a yeast GCN4 leucine zipper DNA-binding motif, e.g. a binding motif forming homodimers, a binding motif forming homotetramers, or preferably a binding motif forming homotrimers; and a binding site for a chemical inducer of dimerization (CID) molecule, such as AP1903.
Embodiment 7: The nucleic acid molecule of any one of Embodiments 1 to 6, wherein the at least one cytokine is linked to the transmembrane region by at least one of (i) a flexible linker such as that defined in SEQ ID Nos: 3-4 or 5-6; (ii) a membrane-proximal region of the ectodomain of an integral membrane protein such as a heavy chain of HLA-A2 (HLA-bridge); and (iii) a carboxy-terminal flanking cysteine-rich region (CFR) of a leucine-rich repeats (LRR) motif-containing protein, such as TLR4.
Embodiment 8: The nucleic acid molecule of any one of Embodiments 1 to 7, wherein the cytoplasmic region comprises more than one signalling element, and the signalling elements are linked to each other by at least one flexible linker.
Embodiment 9: The nucleic acid molecule of any one of Embodiments 1 to 8, wherein the nucleic acid is selected from RNA, such as mRNA, and DNA.
Embodiment 10: The nucleic acid molecule of Embodiment 9, wherein the nucleic acid is DNA, and the nucleotide sequence further includes at least one T cell activation control element, such as a nuclear factor of activated T-cells (NFAT)-responsive promoter, an NF-KB promoter, an IL-2 promoter, or an IL-2 receptor (IL-2R) promoter, which controls the expression of the polypeptide.
Embodiment 11: A vector comprising the nucleic acid molecule of any one of Embodiments 1 to 10, the vector preferably being a retroviral vector.
Embodiment 12: An immune cell comprising the nucleic acid molecule of any one of Embodiments 1 to 10, or the vector of Embodiment 11.
Embodiment 13: The immune cell of Embodiment 12, that is a CD4+ T cell.
Embodiment 14: The nucleic acid molecule of any one of Embodiments 1 to 10, the vector of Embodiment 11, or the immune cell of Embodiment 12 or 13, for use in treating a disease, disorder or condition manifested in aberrant excessive activity of the immune system, such as an autoimmune or inflammatory disease or disorder or condition.
Embodiment 15: A method for treating a disease, disorder or condition manifested in aberrant excessive activity of the immune system, such as an autoimmune or inflammatory disease or disorder or condition, comprising administering to a subject in need thereof the nucleic acid molecule of any one of Embodiments 1 to 10, the vector of Embodiment 11, or the regulatory immune cell of Embodiment 12 or 13.

### Examples

### Materials and Methods

Antibodies: Rat anti-human IL-10 APC, mouse anti-Hhuman CD3 (OKT3) from Miltenyi Biotec. Mouse monoclonal antibodies against human CD4 Alexa Fluor 405, CD279 (PD1) Alexa flour700 and CD49b PE from R&D systems. Mouse anti-human CD223 (LAG-3) PerCP-eFluor 710 and CD4 eFluor 450 from Invitrogen. Mouse monoclonal antibodies against human CD49b PE and CD223 (LAG-3) BV421 from BD biosciences. Purified BB7.2 (Anti-human HLA-A2) from the lab; Goat anti mouse IgG Alexa Fluor 647 from Abcam.

Cells: Melanoma 579 (HLA-A2-) and melanoma 579-A2 (HLA-A2+) were cultured in DMEM supplemented with FBS, pen-strep antibiotic, sodium pyruvate (DMEM complete medium-C.M.). HEK293T is a human embryonic kidney cell line. This cell line is a highly transfectable derivative of HEK 293 cells and contains the SV40 T-antigen. These cells grew in DMEM C.M. Human PBMC's (including CD4 T cells) from healthy donors (#101090621 and #116250421) were cultured in T cell complete medium consisting of RPMI 1640 supplemented with heat-inactivated FBS, glutamax, hepes, βME, pen-strep antibiotic, sodium pyruvate and recombinant human IL-2.

Cloning plasmids and expression vectors: pGEM4Z-GFP-64A plasmid contains 64 A-T bp poly adenosine tail and added restrictions sites. mRNA transcription is controlled by a bacteriophage T7 promoter. This vector is used for mRNA in-vitro synthesis. pQCXIN-eGFP plasmid is a self-inactivating bicistronic retroviral vector for expression of a gene together with a neomycin-resistance marker.

TABLE 2
Primers: The primers were ordered through
IDT (Syntezza Bioscience Ltd).

| Primer | | SEQ ID |
|---|---|---|
| no. | DNA Sequence | NO: |
| 1591 | TATCCAGCCCTCACTCCTT | 83 |
| 1681 | CCTCTTGGTGCTGGTCTTTATC | 84 |
| 1682 | ATTGGAACGCTGCAGGAA | 85 |
| 1683 | AGTGGTGAGGGTAGCACTAA | 86 |
| 1684 | ATTGCTGGCCTGGTTCTC | 87 |
| 1689 | CTCGTTTAGTGAACCGTCAGAT | 88 |

Production of in vitro transcribed mRNA: Template DNA for in-vitro RNA transcription was available in pGEM4Z. The plasmids were linearized using Spel restriction enzyme and precipitated in ammonium acetate and ethanol prior to the experiment. Two stages of transcription were conducted in a final 100 ul reaction mix at 37° C. using a commercial kit to generate 5′-capped in vitro transcribed mRNA. Purification of mRNA was performed by DNase-I digestion and stopped by LiCl and cold UPW, according to the manufacturer's instructions. The mRNA was cleaned with ethanol, its quality was checked by agarose gel electrophoresis, and concentration was determined by a Nanodrop device. The mRNA was stored at −80° C. in small aliquots for use in the capping stage (stage II) on the next day.

PBMC isolation and activation: Human PBMCs were obtained from the MDA National Blood Services (Tel-Hashomer) and from ‘Cell Generation’, BioPark, Hadassah Ein Kerem, Jerusalem. Whole blood samples (or apheresis) were separated using Ficoll-Paque density gradient centrifugation, the PBMCs were collected and washed several times with PBS. PBMCs were then allowed a 24-hour rest in complete RPMI1640 culture medium or were frozen in NutriFreez® D10 Cryopreservation Medium (Biological Industries, Beit Haemek, Israel). Human lymphocytes were cultured in complete RPMI 1640 medium supplemented with 1% (w/v) nonessential amino acids, 25 mM HEPES, 50 mM 2-ME mercaptoethanol, and 1,000 IU/ml recombinant human IL-2 (rhIL-2; Chiron, Amsterdam, The Netherlands). Twenty four hours post-separation (or after cell thawing) PBMCs were activated for 72 hours by plate-bound anti-CD3 Ab (OKT3) in the presence of soluble anti-CD28 and recombinant human IL-2. CD4 T cells were then separated using positive selection with magnetic beads (BD IMag™) and then placed in complete medium for a 24 hour rest before experimental use.

Assembly of clones: The constructs were assembled using standard gene cloning protocols.

PBMC activation and CD8+/CD4+ T cell separation: PBMCs were placed at a concentration of 1.5×10⁶ cells/ml in growth medium containing 250 ng/ml OKT3, 125 ng/ml anti-CD28 mAb and 1,000 U/ml rhIL-2 and incubated for 72 hours. Once showing cluster formation, cells were harvested and thoroughly washed. Separation of CD4+ cells was performed using magnetic beads (IMagTM Anti-Human CD4+Magnetic Particles, BD Biosciences, San Jose, CA) according to the manufacturer's protocol and separated cells were cultured in the presence of 1,000 U/ml rhIL-2.

In-vitro transcription of mRNA: Template DNA for in-vitro mRNA transcription was cloned into the pGEM4Z/GFP/A64 vector (Boczkowski et al., 2002, Cancer. Res. 60:1028-34) following removal of the GFP insert. Plasmids were linearized using the Spel restriction site and subjected to in-vitro transcription reaction in a 20 ul reaction mix at 37° C. using the “AmpliCap-MaxTM T7 High Yield Message Maker Kit” (T7 mScript Standard mRNA Production System, CELLSCRIPT, Madison, WI) to generate 5′-capped mRNA. Purification of mRNA was performed by DNase-I digestion, followed by LiCl precipitation according to the manufacturer's instructions. The mRNA quality was confirmed by agarose gel electrophoresis and concentration was determined by a NanoDrop spectrophotometer (Thermo Fisher Scientific, Waltham, MA). RNA was stored at −80° C. in aliquots.

mRNA transfection through electroporation of human CD4 T cells: Activated CD4 T cells were washed with Opti-MEM medium. After counting the cells by using Trypan Blue dye and Countess 3 Automated cell counter, they were resuspended in Opti-MEM at a final concentration of 22.2×10⁶ cell/ml. Cells and cuvettes were pre-chilled on ice before electroporation. 180 μl of the cells was mixed with 10 ug mRNA and electroporated in 2-mm cuvettes using Gene Pulser Xcell electroporation, with Square Wave mode, 360V, 1 ms, 4 ×10⁶ cells, one pulse. Immediately after electroporation, the cells were transferred to a fresh growth medium.

T cells stimulation through CAR signaling-co-culture with Melanoma: After 6 hours of incubation of the transfected T cells, 500K cells of Melanoma 579 (HLA-A2-) and 579-A2 (HLA-A2+) were collected from their medium, using trypsin-EDTA for cessation of cell adhesion, for co-culture with the transfected T cells on a 1:1 ratio. The cells were incubated overnight.

T cells stimulation through TCR signaling: As an alternative for the stimulation of the transfected T cells by co-culturing them with Melanoma, we performed overnight incubation of the cells with 30 ng/ml of a-CD3 for TCR activation.

Flow analysis of cell surface expression of membrane cytokines constructs and other markers. The treated T cells were harvested, washed once with 3 ml cold FACS buffer (500 ml PBS with 500 μl EDTA, 2.5 ml sodium azide and 1 g BSA) and incubated for 10-40 minutes at room temperature in the dark with the respective Ab-conjugate at the concentration recommended by the manufacturer. Cells were washed twice again with 3 ml and 2 ml FACS buffer, resuspended in 0.4 ml PBS and subjected to flow analysis using NovoCyte Flow Cytometer System and data was analyzed using NovoExpress Software.

In-vitro functional analysis of membrane cytokines: Functional analysis was used to assess the effect of the membrane cytokines on the upregulation of Tr1 phenotype. The production of IL-10 and IFN-γ was assayed by a commercial ELISA kit.

Production of competent cells and transformation: Top 10 E. coli cells were grown in liquid LB in shake at 37° C., 167 rpm, and once achieving an O.D. of 0.4-0.6 nm according to the spectrophotometer reading, the cells were submerged in the centrifuge for 10 min. at 4° C., 7000 rpm. The pellet was re-suspended in 1 ml 0.1M sterile cold filtered Calcium Chloride.

Transformation was done by taking 5 μl DNA into 100 μl competent cells. The mixture was placed into ice for 30 min., transferred to a heated block at 42° C. for 2 min., and then returned to the ice for 20 min. 500 μl of liquid LB was added and then the mixture was incubated for 1 hour at 37C, 160 rpm. Following, the cells were centrifuged at 7000 rpm for 5 min., the supernatant discarded, and the pellet was re-suspended in 100 μl LB. The bacteria spread on LB agar plates containing selective medium (CARB antibiotic), then were incubated at 37° C. overnight and then transferred to 4° C. on the day after. Bacterial colonies were chosen and collected into tubes containing 1:1000 Ampicillin and LB. The tubes were incubated in shake at 37° C., 160 rpm overnight.

DNA digestion, ligation, and agarose gel analysis: During the experiments, analytical 1% agarose gel electrophoresis (0.6 g agarose, 60 ml buffer TAE and two drops of Ethidium bromide) was used to confirm segments lengths and purity, and concentrations were evaluated using a Nanodrop device. DNA was digested using the restriction enzymes-BamH1-HF and EcoR1-HF and 10% Cutsmart buffer overnight at 37° C. After assuring the digestion, the DNA segments were run and separated on 1% (0.8 g agarose, 80 ml buffer TAE and two drops of Ethidium bromide) agarose gel with TAE buffer and 200 μl crystal violet. The desired bands were cut from the gel and cleaned using a commercial kit. Ligation of the DNA inserts into their vectors was done by using 1 unit of T4 DNA Ligase at 37° C. for 1 hour and then at 4° C. overnight.

DNA production: Four ml of the transformed bacteria were centrifuged and then treated to extract the DNA in a small amount according to the kit manufacturer's instructions. Samples were sent to Hylabs for sequencing, and after assuring the sequence, production of the plasmids in a big quantity was done using a commercial kit.

DNA transfection-Virion synthesis: Forty-eight hrs. prior to the DNA transfections Hek293 cells were collected and plated on Poly-D-Lysine coated plates at a concentration of 150k-300k cells/ml. Cells are then transfected with jetOPTIMUS transfection reagent, gene of interest plasmid and packaging plasmids (gag/pol, env) on a 3:3:2 ratio, respectively. The reagent/DNA mixture was added dropwise onto the Hek293 cells.

Retroviral transduction: Twenty-four hours prior to the T cell transduction, 6-welled noncoated cell cultured plates were coated with RetroNectin (20 ug/ml). Viral supernatant was added to the RetroNectin coated plates and centrifuged for 2 hrs. at 2000 G. The supernatant was then mostly removed and CD4+ T cells were added to the wells. Cells were then centrifuged at 1500 rpm for 10 minutes without acceleration and brake on. 24 hrs. post transduction cells were transferred to standard cell culture plates and rested for 72 hrs. prior to analysis.

Statistical analysis: Statistical analyses were performed using GraphPad Prism V.9.3.1 software, using a one-way analysis of variance followed by the Tukey's multiple comparison test. Significance levels are indicated in the figures *p<0.05; ** p<0.01; *** p<0.001 ns-non-significant. Graph bars present the differences in the activity of the study treatments and presented as mean±SEM.

### Example 1: Design of memIL-10-CD40 Constructs

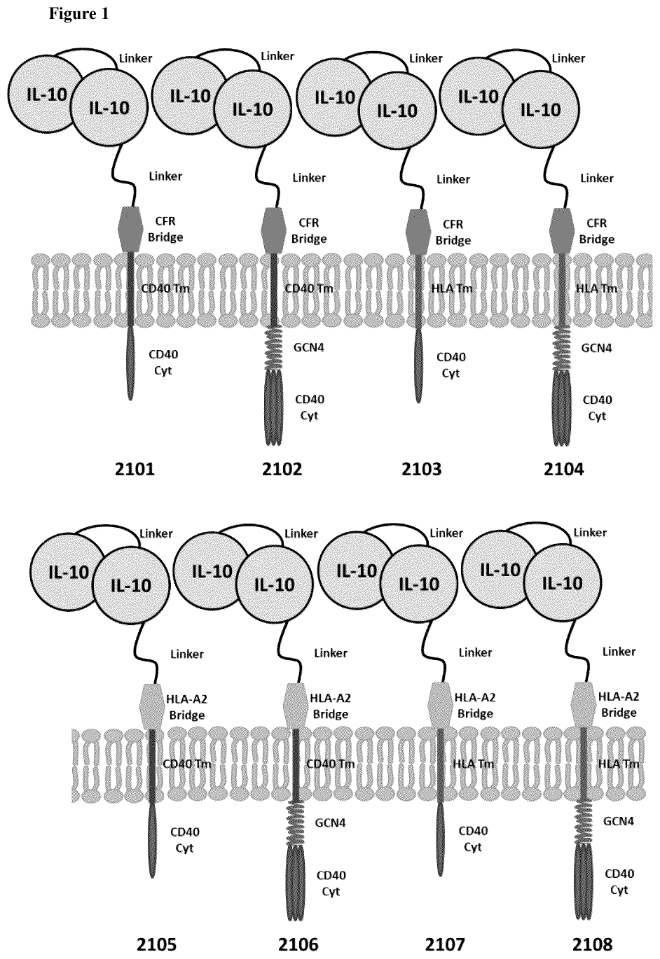

First, a tandem single-chain IL-10 (scIL-10, SEQ ID NO: 2) was prepared from two IL-10 monomers (SEQ ID NO: 1) linked together in tandem by a flexible linker (GSTSGSGKPGSGEGSTKG, in SEQ ID NO: 3) to create a homodimer. A signal peptide was added to produce surface expression (SEQ ID NO: 4) although it will be understood that the final protein product scIL-10 would be devoid of the signal peptide (SEQ ID NO: 5). To prepare membranal IL-10 (memIL-10, FIG. 1B, clone 1354, SEQ ID NO: 6), the previously described design of membrane cytokines was followed (Weinstein-Marom et al., 2016, “Membrane-attached Cytokines Expressed by mRNA Electroporation Act as Potent T-Cell Adjuvants”, J Immunotherapy; 39:60-70, the contents of which are hereby incorporated by reference in their entirety). This was accomplished by linking the single-chain tandem IL-10 to a transmembrane-intracellular stretch via a flexible hinge region having a 21 amino acid spacer comprising the short flexible linker Gly4Ser (Gly3Ser) 2 (SEQ ID NO: 7) and an additional 8 amino acid bridge of the sequence SSQPTIPI (SEQ ID NO: 8) derived from the membrane-proximal part of the connecting peptide of HLA-A2.

It was hypothesized that memIL-10 function could be significantly enhanced by the signaling of CD40, as previously shown for the membrane-bound cytokines memIL-2, memIL-12 and memIL-15 (Weinstein-Marom et al., 2016 and Weinstein-marom et al., 2019, “Combined Expression of Genetic Adjuvants Via mRNA Electroporation Exerts Multiple Immunostimulatory Effects on Antitumor T Cells: J. Immunotherapy, 42:43-50, the contents of which are hereby incorporated by reference in their entirety). To assure equal stoichiometry of the different protein constituents, an ‘all-in-one’ synthetic transmembrane molecule with the components arranged in tandem on the same molecule was assembled. To this end scIL-10 (SEQ ID NO: 2) was tethered by joining the linker to the ectodomain C-terminal flanking cysteine-rich region (CFR) bridge (SEQ ID NO: 9) or to the HLA-A2 bridge (SEQ ID NO: 10), to a CD40 or HLA-H2 transmembrane domain (SEQ ID NO: 11 or SEQ ID NO: 12, respectively), and finally to a GCN4-CD40 trimer unit (SEQ ID NO: 13, caCD40), or a CD40 monomeric domain (SEQ ID NO: 14). By combining various permutations of these modules eight memIL-10-CD40 constructs were created (FIG. 1, Table 3).

TABLE 3
constructs used

| Clone number | SEQ ID NO | construct |
|---|---|---|
| 1655 | — | Irr.mRNA, negative control |
| 1354 | 6 | memIL-10, positive control |
| 2101 | 15 | memIL-10 (CFR-CD40)-CD40(mon) |
| 2102 | 16 | memIL-10 (CFR-CD40)-CD40(tri) |
| 2103 | 17 | memIL-10 (CFR-HLA-A2)-CD40(mon) |
| 2104 | 18 | memIL-10 (CFR-HLA-A2)-CD40(tri) |
| 2105 | 19 | memIL-10 (HLA-A2-CD40)-CD40(mon) |
| 2106 | 20 | memIL-10 (HLA-A2-CD40)-CD40(tri) |
| 2107 | 21 | memIL-10 (HLA-A2)-CD40(mon) |
| 2108 | 22 | memIL-10 (HLA-A2)-CD40(tri) |

### Example 2: Transient Expression Evaluation Through mRNA Transfection

First, transient expression of the constructs as mRNA was carried out in CD4 T cells, for a simple and fast primary test, as a base for the preparation of a viral vector of the most promising construct. Of note, it was assumed that transient and short-term expression is not enough to push the cells to differentiate towards Tr1 cells, so it is doubtful whether the expected function will fully express in this experiment under these conditions. The DNA of the eight constructs (SEQ ID NO: 23-30) was available and two stages of mRNA production was performed from the constructs of the invention (2101-2108) and the control constructs (1354—an inert anchored memIL-10 without a costimulatory intracellular component as a reference for solely memIL-10 function and 1655-irrelevant mRNA for a negative control) according to the manufacturer's protocol. The HLA-A2 CAR gene (1558) was available after stage 1 production; therefore, only the stage 2 procedure was performed. The mRNA segments were run on agarose gel for quality check, concentrations were evaluated using a Nanodrop device and the samples were divided into new test tubes for the continued electroporation containing 10 μg of mRNA.

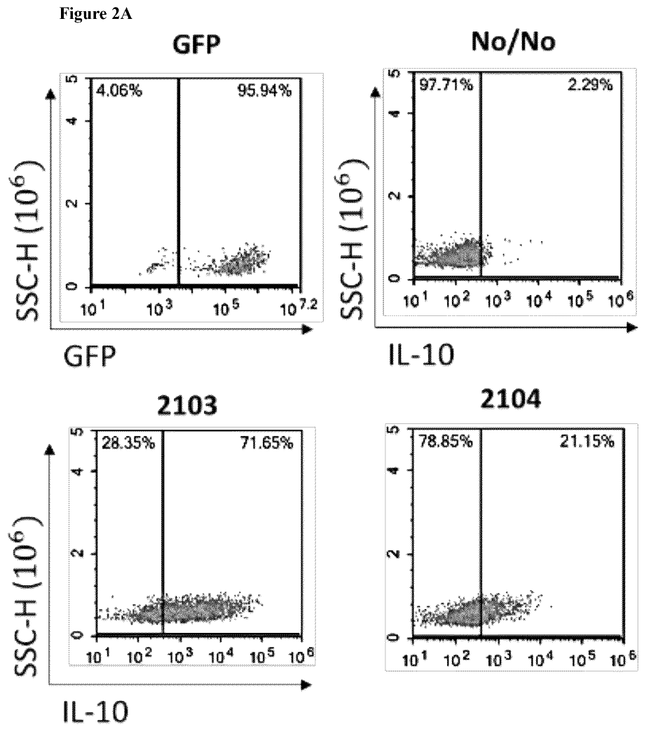

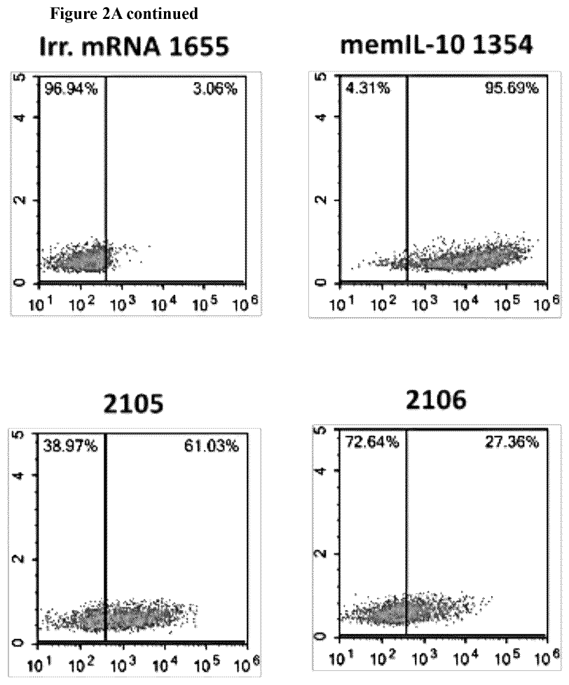

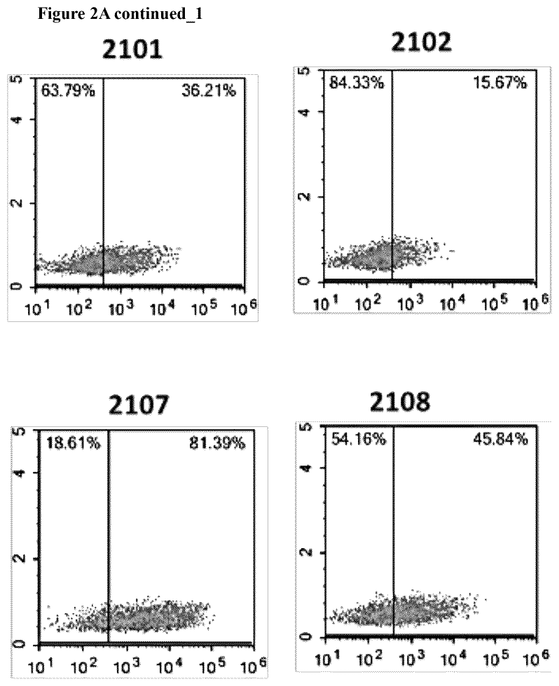

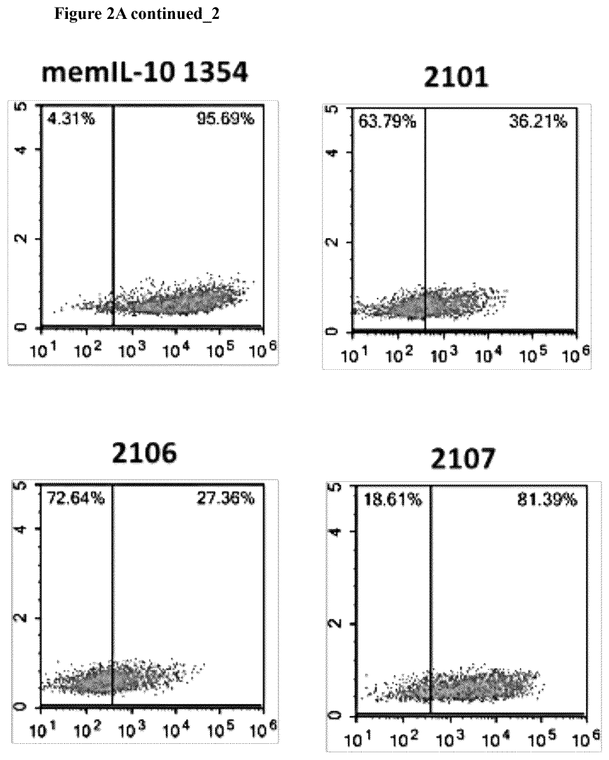

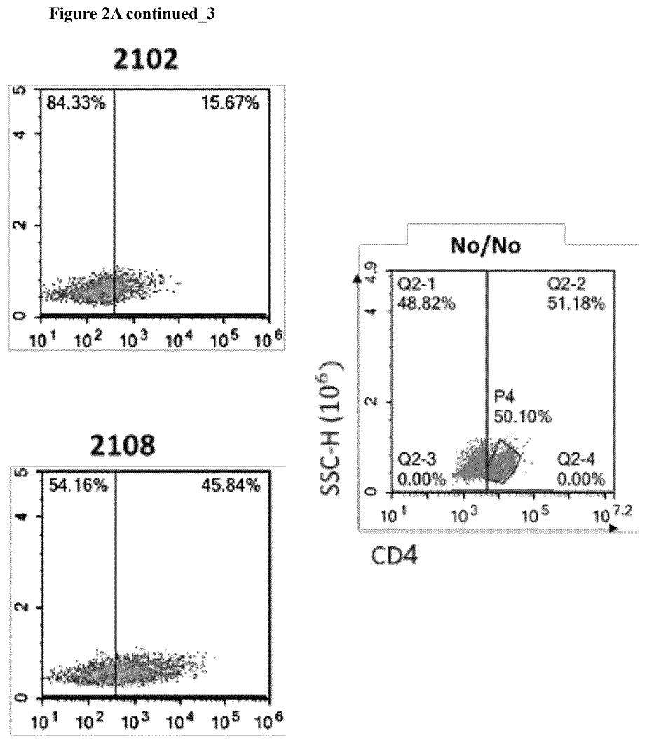

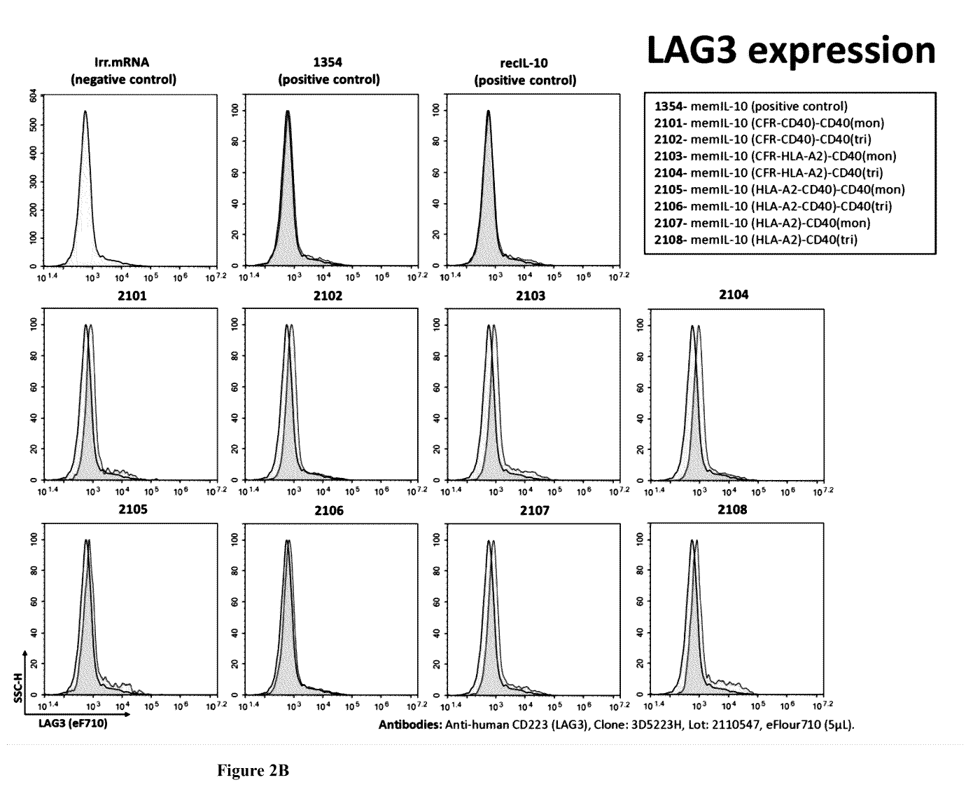

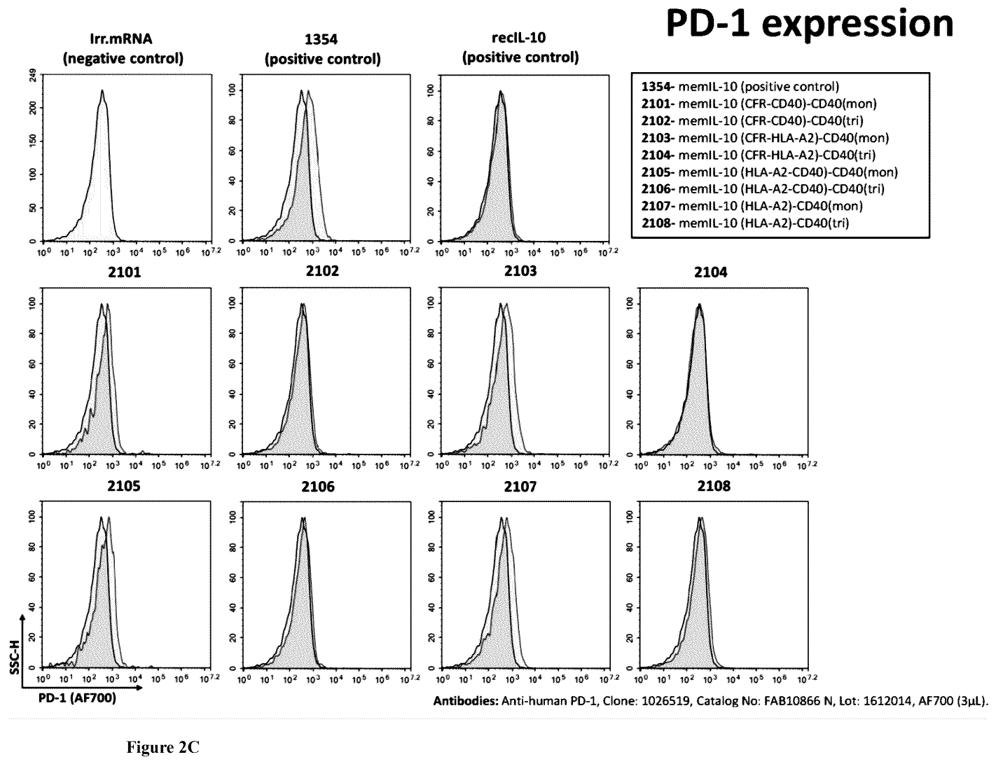

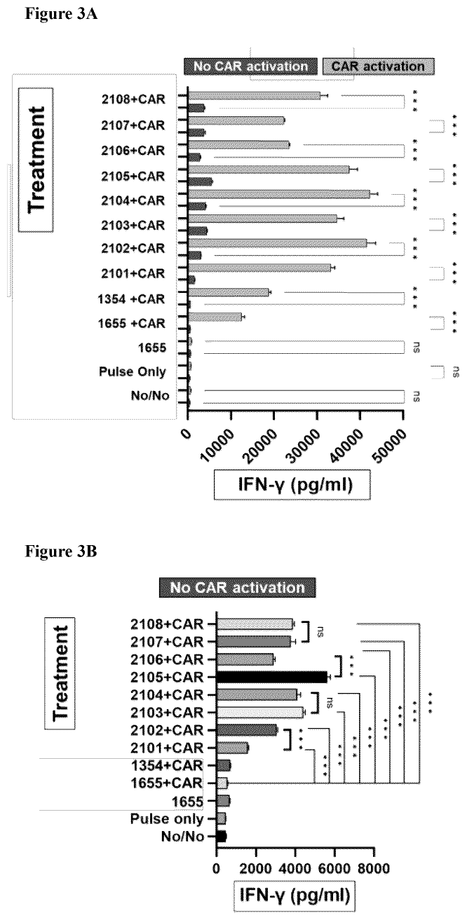

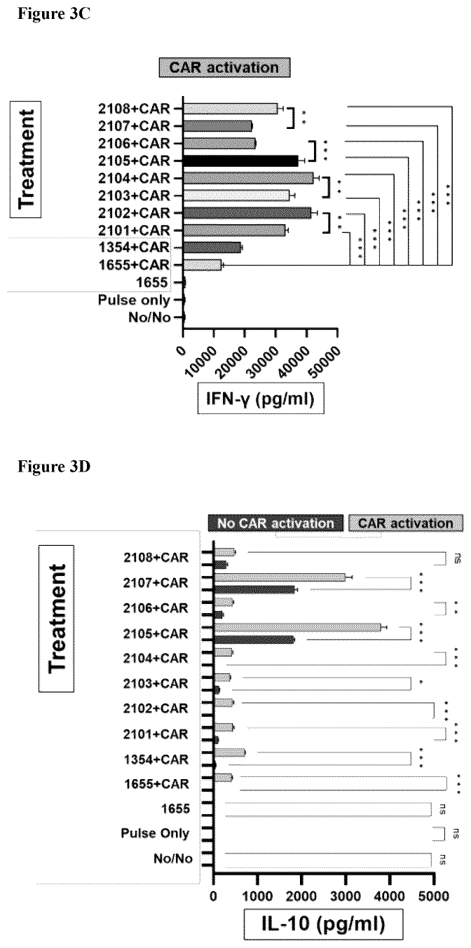

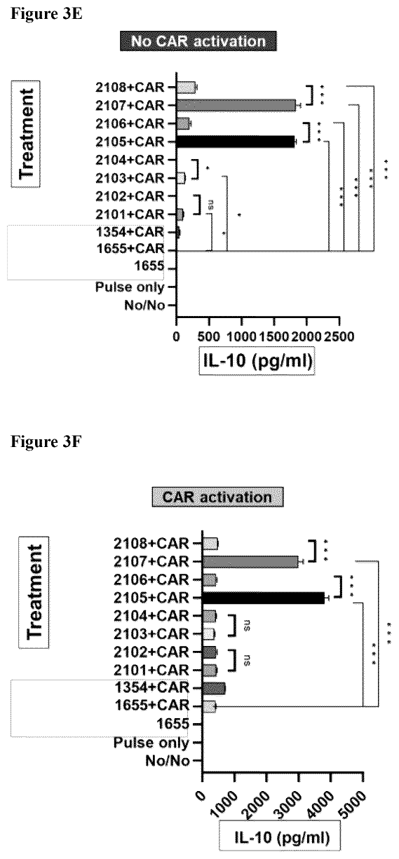

Transfection through mRNA electroporation of human CD4 T cells (Negative fraction of cells after positive selection for CD8 from total PBMC) was performed with the synthesized mRNA of the adjuvants (constructs) and the HLA-A2 CAR and control genes (Transfection #1). The transfected cells were then co-cultured with Melanoma 579-A2, and 579 as a control, to examine the induced T cell's function after CAR activation. The T cells were collected and evaluated for memIL-10 (FIG. 2A), and the CD49b, LAG-3 (FIG. 2B), and PD1 (FIG. 2C) markers expression through Flow Analysis. Expression of the LAG3 and PD-1 Tr1 markers (as determined using an eFlour710-conjugated anti-human CD223 (LAG3) mAb and an AF700PE-conjugated anti-human PD-1 mAb) is detected in CD4+ T cells, indicating activation of the corresponding pathways. The low level of activation is likely due to the use of transient transfection. Supernatant was likewise collected for performing an ELISA test for secretion of IL-10 and IFN-γ (FIG. 3).

As shown in FIG. 2A the GFP results indicate high efficacy of the transfection (here >95%). The results showed good expression of the IL-10 marker in comparison to the negative control (irrelevant mRNA-1655), while the monomer CD40 constructs (2101, 2103, 2105, 2107) showed better expression than their corresponding trimer CD40 constructs (2102, 2104, 2106, 2108, respectively). In a comparison of the bridge components, the HLA-A2 bridge (in constructs 2105, 2106, 2107, 2108) contributes to the expression more than the CFR bridge in constructs (2101, 2102, 2103, 2104, respectively). Also, the constructs which contain the HLA Tm (2103, 2104, 2107, 2108) were expressed better than those with the CD40 Tm (2101, 2102, 2105, 2106, respectively). The expression of other markers (CD49b, LAG-3, PD1) was not different than the expression in the negative control and the CAR activation did not show any impact on the cells' marker expression (both results are not shown). The results for the CD4 marker showed, as expected, that there were two populations of cells-positive and negative for CD4, since we used an impure population of CD4 T cells.

The ELISA test results (FIG. 3A-3F) presented high secretion of IL-10, despite the use of impure population of CD4 T cells. As shown in FIG. 3D, there is a distinct increment in the secretion of IL-10 with CAR activation in most treatments. Without the CAR activation (FIG. 3E), most monomer CD40 constructs (2103, 2105, 2107) secreted a higher level of IL-10 than their trimer analogized constructs (2104, 2106, 2108), while with CAR activation (FIG. 3F), only half of the monomer constructs (2105, 2107) secrete higher levels than their analogized constructs (2106, 2108). Without CAR activation, the HLA-A2 bridge (2105, 2106, 2107, 2108) secretes distinctly higher levels of IL-10 than the control, while the CFR bridge constructs (2101, 2102, 2103, 2104) do not. With the CAR activation, only the HLA-A2 bridge/monomer CD40 constructs (2105, 2107) showed distinctly higher levels of IL-10 than the control. All cells showed a basal level of IFN-γ secretion (FIG. 3B) and a distinct increase in secretion after CAR activation (FIG. 3A). Most constructs showed higher secretion of IFN-γ than the positive control, likely due to the CD40 intracellular component. After CAR activation (FIG. 3C), most trimer CD40 constructs (2102, 2104, 2108) showed a higher level of IFN-γ secretion than the monomer CD40 constructs (2101, 2103, 2107).

To summarize, good expression of the mRNA of the adjuvants/constructs was observed, assuring their decent production, and demonstrating their enhancement potential. The high levels of IFN-γ secretion indicate that the CAR activation was possibly too strong. Hence, we proceeded to another experiment on a purer population of cells, this time using TCR activation.

In a similar experiment (Transfection #2), separated human CD4 T cells were transfected through mRNA electroporation with the constructs of the invention and then performed a TCR activation assay by incubating the transfected T cells with an a-CD3 mAb (OKT3). Cells were again collected and evaluated through Flow Analysis.

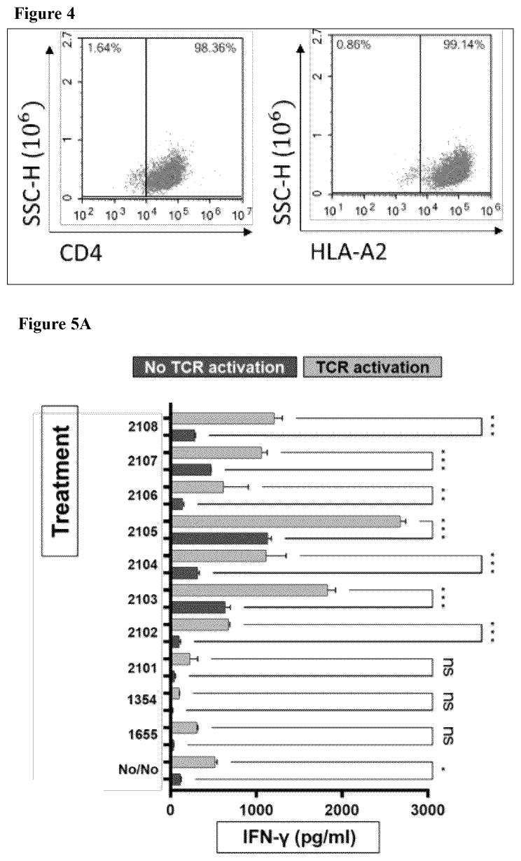

Before the experiment, the expression of CD4 and HLA-A2 in the cell population was examined through Flow Analysis to detect the cells' activation manner. The results indicated that the population of pure CD4 T cells are positive for HLA-A2 (FIG. 4). Hence, it was decided to activate them through TCR activation and not CAR activation, otherwise, the activation could be caused by the CD4 cells themselves, regardless of the co-culture with positive melanoma cells.

The second transfection has shown similar expression results, also the expression of other markers was not different than the expression in the negative control, and no effect of the TCR activation on the cells' marker expression was observed, therefore they are not presented here.

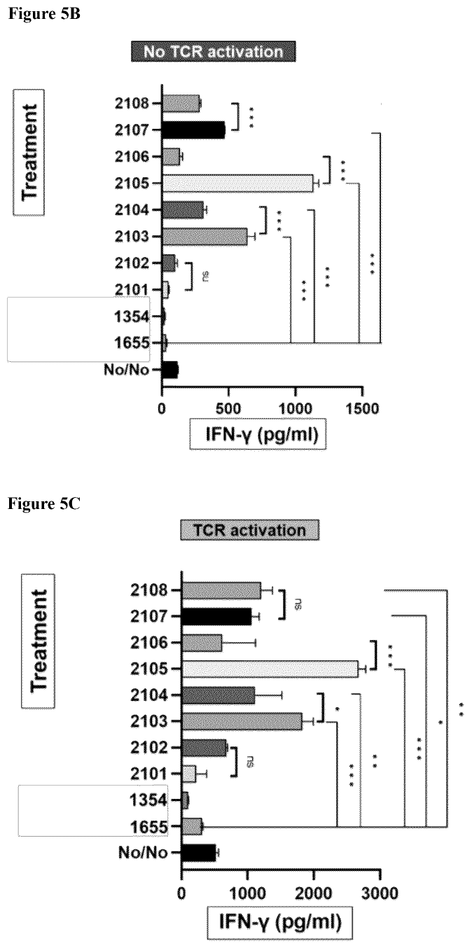

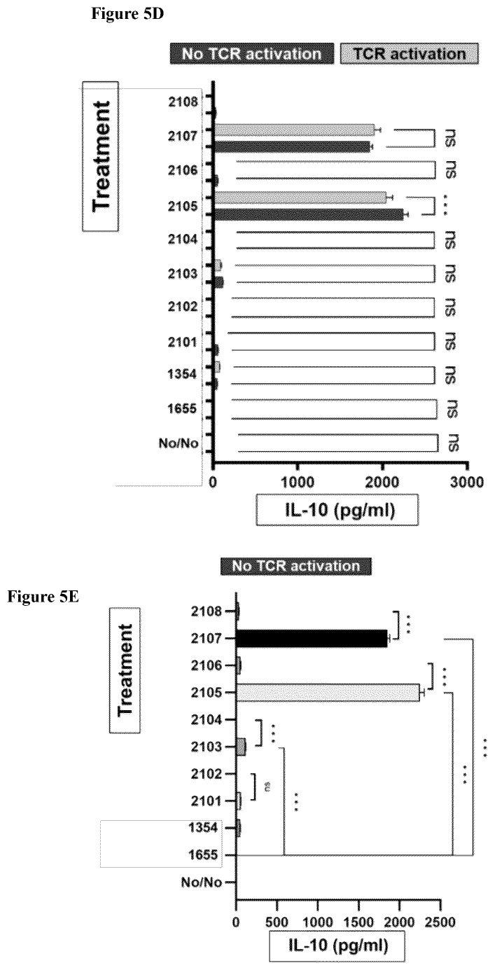

Growth medium was likewise collected for performing an ELISA test for the secretion of IL-10 and IFN-γ. As seen in FIGS. 5A-5F, the TCR activation impacts mainly the secretion of IFN-γ (FIG. 5A) but also the secretion of IL-10 by construct 2105 (FIG. 5D). The results also showed a high secretion of IL-10 from the T cells transfected with monomer construct (2103, 2105, 2107) compared to the negative and positive control, but low or non-secretion from the cells transfected with the trimer constructs (2104, 2106, 2108) (FIG. 5E-5F). As for the secretion of IFN-γ, both with and without TCR activation (FIG. 5C-5B, respectively), the constructs 2103, 2104, 2105, 2107, and 2108 showed higher secretion than the control. While after activation most monomer CD40 constructs (2103, 2105, 2107) secreted a higher level of IFN-γ than their corresponding trimer constructs (2104, 2106, 2108).

The two transfection experiments motivated us to select the most suitable constructs-2105, 2107 (that showed the higher IL-10 secretion and expression and decent levels of IFN-γ secretion) and their corresponding trimer constructs 2106, 2108, to proceed with them for a stable expression experiment.

### Example 3: Transient Expression Evaluation Through mRNA Transfection

To use constructs 2105-2108 in viral transduction, a standard cloning process was performed in which the genes were transferred from the RNA vector-PGEM4Z (2105-8) into a suitable vector for retroviral transduction-pQCXIN (991) by using the restriction enzymes BamH1-HF and EcoR1-HF.

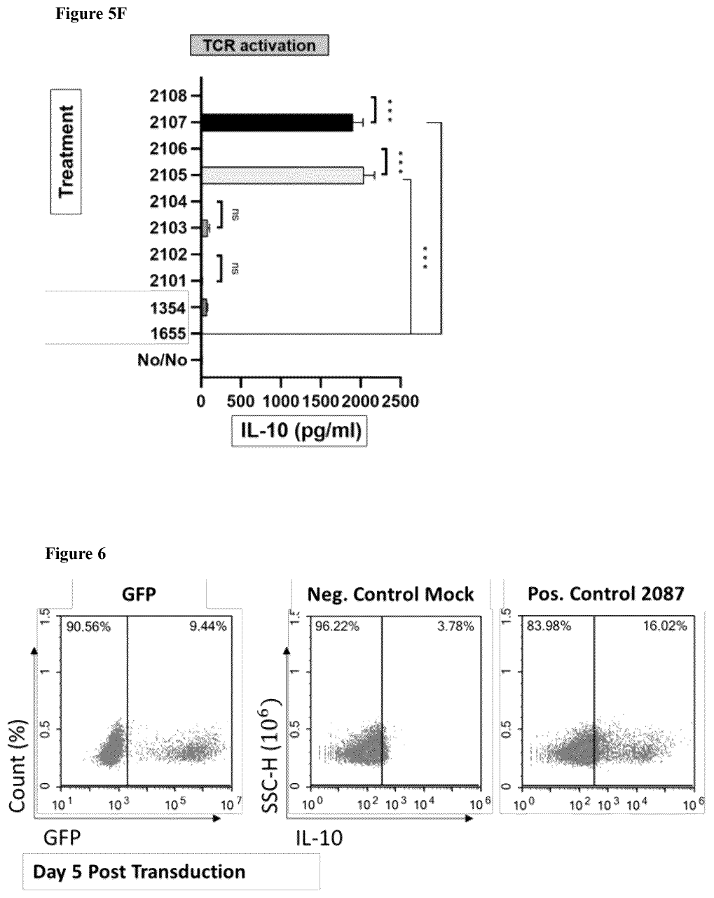

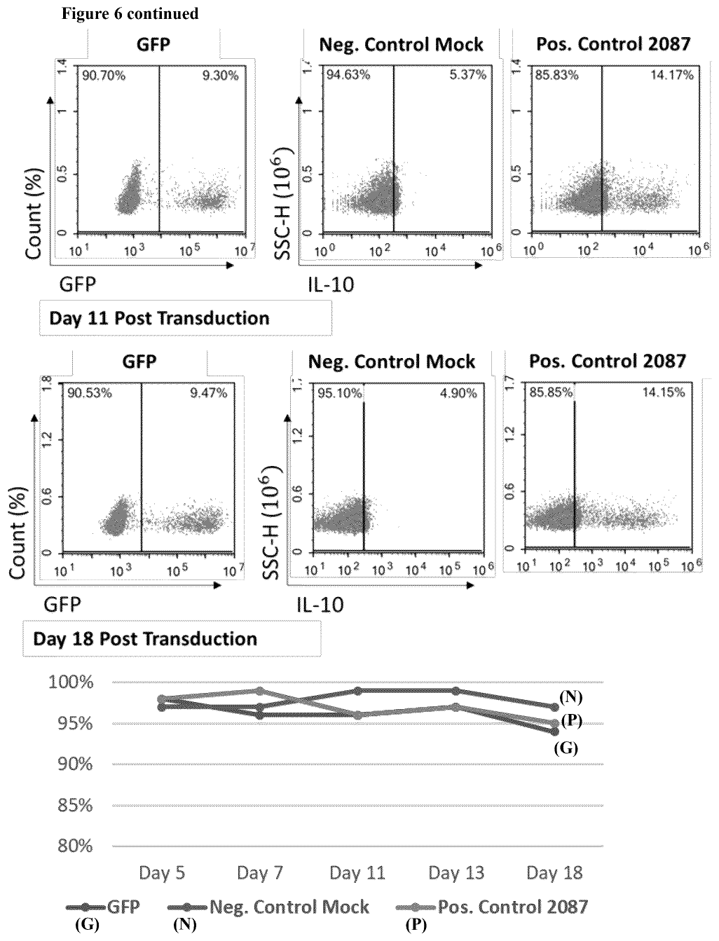

Simultaneously with the cloning process, a calibration experiment for the transduction system was conducted in which retroviral transduction of CD4 T cells was performed with control vectors only. Cells were collected and evaluated through Flow Analysis over three weeks. As shown in FIG. 6, the transduction efficacy as judged by the GFP vector was ˜9% and the positive control expressed higher (˜15%). The survival rate of cells was consistent.

### Example 4. Assessing Inhibitory Effect of Transduced Cells on T Effector Cells

To examine the ability of transduced cells to exert their inhibitory effect on neighboring Teff cells a coculture setting is designed which will allows the selective activation of only one T cell population and not the other (obviously, anti-TCR/CD3 antibodies would activate all T cells in the coculture). To this end, two genes encoding the chimeric H-2K^{b}-CD35 (K^{b}-CD3ζ) and H-2K^{d}-CD3ζ(K^{d}-CD3ζ) MHC-I heavy chains are employed. Both genes selectively activate T cells following Ab-mediated cross-linking in magnitude that is comparable to TCR cross-linking. These tools are employed to mix mRNA-transfected Tr1 and Teff cells at different ratios for 3-4 days and carboxifluorescein diacetate succinimidyl ester (CFSE) dilution and intracellular IFN-γ staining are used to assess the ability of activated Tr1 cells (vs. non-activated or Red Fluorescence Protein (RFP)+non-Tr1 cells) to suppress both proliferation and effector function of the activated Teffs.

1. A nucleic acid molecule comprising a nucleotide sequence encoding a chimeric polypeptide comprising:
a. a cytokine region comprising at least one single-chain anti-inflammatory cytokine;
b. a transmembrane region; and
c. at least one signaling region comprising an element of a T cell costimulatory receptor of the tumor necrosis factor receptor (TNFR) family protein.

2. The nucleic acid molecule of claim 1, wherein at least one of:
a. said single chain anti-inflammatory cytokine is a single chain interleukin 10 (IL-10);
b. said cytokine region comprises a tandem repeat of said single chain anti-inflammatory cytokine, wherein said tandem repeats are linked by an amino acid linker; and
c. said cytokine region comprises a tandem repeat of single chain IL-10, linked by an amino acid linker.

3. The nucleic acid molecule of claim 2, wherein said single chain IL-10 comprises SEQ ID NO: 1, said tandem repeat of single chain IL-10 comprises SEQ ID NO: 5 or both.

4. (canceled)

5. (canceled)

6. (canceled)

7. The nucleic acid molecule of claim 1,
wherein the transmembrane region comprises a transmembrane domain of a single pass transmembrane protein, and wherein said protein is selected from an human leukocyte antigen A (HLA-A), an HLA-B, an HLA-C, CD40, Toll-like receptor 4 (TLR4), and CD28, optionally wherein HLA-A is HLA-A2.

8. (canceled)

9. (canceled)

10. The nucleic acid molecule of claim 7, wherein said HLA transmembrane domain comprises SEQ ID NO: 12, said CD40 transmembrane domain comprises SEQ ID NO: 11 or both.

11. The nucleic acid molecule of claim 1, wherein the T cell costimulatory receptor of the TNFR family protein is selected from a CD40, CD27, 4-1BB (CD137), OX40 (CD134), herpesvirus entry mediator (HVEM/TNFRSF14), CD30, and glucocorticoid-induced TNFR-related protein (GITR).

12. The nucleic acid molecule of claim 11, wherein said T cell costimulatory receptor of the TNFR family is CD40, said signaling element comprises SEQ ID NO: 14, or said signaling region comprises a trimeric CD40 signaling element comprising SEQ ID NO: 13 or both.

13. (canceled)

14. The nucleic acid molecule of claim 1, wherein at least one of:
a. said signaling region further comprises at least one self-assembly domain;
b. said polypeptide further comprises a membrane-proximal region selected from SEQ ID NO: 8, 9 and 10;
c. said regions are directly linked by amino acid linkers or peptide bonds; and
d. said polypeptide further comprises a signal peptide.

15. The nucleic acid molecule of claim 14, wherein the at least one self-assembly domain is a coiled coil domain from a yeast GCN4 leucine zipper DNA-binding motif, optionally wherein said GCN4 coiled coil domain comprises SEQ ID NO: 31 or SEQ ID NO: 68.

16. (canceled)

17. (canceled)

18. (canceled)

19. (canceled)

20. (canceled)

21. The nucleic acid molecule of claim 1, wherein said polypeptide comprises a sequence selected from SEQ ID NO: 15-30.

22. (canceled)

23. (canceled)

24. An expression vector, comprising a nucleic acid molecule of claim 1 operably linked to at least one transcription regulatory element.

25. The vector of claim 24, wherein at least one of:
a. said vector is an RNA or DNA expression vector;
b. said at least one transcription regulatory element is a T cell active promoter;
c. said at least one transcription regulatory element is a T cell active promoter selected from a nuclear factor of activated T-cells (NFAT)-responsive promoter, an NF-KB promoter, an IL-2 promoter, and an IL-2 receptor (IL-2R) promoter; or
d. said vector is a retroviral vector.

26. (canceled)

27. (canceled)

28. (canceled)

29. A polypeptide encoded by a nucleic acid molecule of claim 1.

30. A cell comprising a nucleic acid molecule of claim 1, optionally wherein said cell is a T cell or a CD4 positive T cell.

31. (canceled)

32. (canceled)

33. (canceled)

34. An enriched population of CD4 T cells, wherein at least 10% of cells in said population comprise a nucleic acid molecule of claim 1.

35. A method of converting a CD4 positive T cell into a regulatory T cell (Treg), the method comprising introducing into said CD4 positive T cell a nucleic acid molecule of claim 1, vector thereby converting a CD4 positive T cell into a Treg, optionally wherein said CD4 positive T cell is a primary cell isolated from peripheral blood.

36. (canceled)

37. (canceled)

38. A pharmaceutical composition comprising the enriched population of claim 34 and a pharmaceutically acceptable carrier excipient or adjuvant.

39. A method for inducing immune suppression in a subject in need thereof, the method comprising administering to said subject a pharmaceutical composition of claim 38, thereby inducing immune suppression.

40. A method for treating a disease, disorder or condition characterized by excessive activity of the immune system or treatable by immune suppression in a subject in need thereof, the method comprising administering to said subject a pharmaceutical composition of claim 38, thereby treating a disease, disorder or condition.

41. The method of claim 40, wherein said disease, disorder or condition is selected from an autoimmune and an inflammatory disease, disorder or condition or wherein said disease, disorder or condition is selected from transplant rejection, cardiovascular disease, obesity, systemic inflammation, celiac disease, dermatomyositis, Graves' disease, Addison's disease, Hashimoto disease, Multiple sclerosis, Crohn's disease, colitis, myasthenia gravis, pernicious anemia, arthritis, Sjogren syndrome, lupus, diabetes, pemphigus vulgaris, pemphigus follicularis, membranous nephropathy, sarcoidosis, vasculitis, atherosclerosis, granulomatosis, spondyloarthropathy and cytokine storm.

42. (canceled)


SEQUENCE LISTING (88 sequences)

SEQ ID NO: 1 - protein, 160 aa, synthetic construct
  REGION 1..160: note = Synthetic
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 356 aa, synthetic construct
  REGION 1..356: note = Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 3 - protein, 18 aa, synthetic construct
  REGION 1..18: note = Synthetic
GSTSGSGKPGSGEGSTKG

SEQ ID NO: 4 - protein, 18 aa, synthetic construct
  REGION 1..18: note = Synthetic
MHSSALLCCLVLLTGVRA

SEQ ID NO: 5 - protein, 338 aa, synthetic construct
  REGION 1..338: note = Synthetic
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 6 - protein, 434 aa, synthetic construct
  REGION 1..434: note = Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGSGGGSSSQPTIPIVGIIAGLVLFGAVITGAVVAAVMWRRKSSDRKGGSYSQAASSDSAQGSDVSLTACKV

SEQ ID NO: 7 - protein, 13 aa, synthetic construct
  REGION 1..13: note = Synthetic
GGGGSGGGSGGGS

SEQ ID NO: 8 - protein, 8 aa, synthetic construct
  REGION 1..8: note = Synthetic
SSQPTIPI

SEQ ID NO: 9 - protein, 13 aa, synthetic construct
  REGION 1..13: note = Synthetic
PVLSLNITCQMNK

SEQ ID NO: 10 - protein, 13 aa, synthetic construct
  REGION 1..13: note = Synthetic
LRWEPSSQPTIPI

SEQ ID NO: 11 - protein, 22 aa, synthetic construct
  REGION 1..22: note = Synthetic
ALVVIPIIFGILFAILLVLVFI

SEQ ID NO: 12 - protein, 24 aa, synthetic construct
  REGION 1..24: note = Synthetic
VGIIAGLVLFGAVITGAVVAAVMW

SEQ ID NO: 13 - protein, 97 aa, synthetic construct
  REGION 1..97: note = Synthetic
HMMKQIEDKIEEILSKIYHIENEIARIKKLIGEELKKVAKKPTNKAPHPKQEPQEINFPDDLPGSNTAAPVQETLHGCQPVTQEDGKESRISVQERQ

SEQ ID NO: 14 - protein, 62 aa, synthetic construct
  REGION 1..62: note = Synthetic
KKVAKKPTNKAPHPKQEPQEINFPDDLPGSNTAAPVQETLHGCQPVTQEDGKESRISVQERQ

SEQ ID NO: 15 - protein, 474 aa, synthetic construct
  REGION 1..474: note = Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLPVLSLNITCQMNKLEALVVIPIIFGILFAILLVLVFIELKKVAKKPTNKAPHPKQEPQEINFPDDLPGSNTAAPVQETLHGCQPVTQEDGKESRISVQERQ

SEQ ID NO: 16 - protein, 507 aa, synthetic construct
  REGION 1..507: note = Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLPVLSLNITCQMNKLEALVVIPIIFGILFAILLVLVFIHMMKQIEDKIEEILSKIYHIENEIARIKKLIGEELKKVAKKPTNKAPHPKQEPQEINFPDDLPGSNTAAPVQETLHGCQPVTQEDGKESRISVQERQ

SEQ ID NO: 17 - protein, 476 aa, synthetic construct
  REGION 1..476: note = Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLPVLSLNITCQMNKLEVGIIAGLVLFGAVITGAVVAAVMWELKKVAKKPTNKAPHPKQEPQEINFPDDLPGSNTAAPVQETLHGCQPVTQEDGKESRISVQERQ

SEQ ID NO: 18 - protein, 509 aa, synthetic construct
  REGION 1..509: note = Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLPVLSLNITCQMNKLEVGIIAGLVLFGAVITGAVVAAVMWHMMKQIEDKIEEILSKIYHIENEIARIKKLIGEELKKVAKKPTNKAPHPKQEPQEINFPDDLPGSNTAAPVQETLHGCQPVTQEDGKESRISVQERQ

SEQ ID NO: 19 - protein, 474 aa, synthetic construct
  REGION 1..474: note = Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLLRWEPSSQPTIPILEALVVIPIIFGILFAILLVLVFIELKKVAKKPTNKAPHPKQEPQEINFPDDLPGSNTAAPVQETLHGCQPVTQEDGKESRISVQERQ

SEQ ID NO: 20 - protein, 507 aa, synthetic construct
  REGION 1..507: note = Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLLRWEPSSQPTIPILEALVVIPIIFGILFAILLVLVFIHMMKQIEDKIEEILSKIYHIENEIARIKKLIGEELKKVAKKPTNKAPHPKQEPQEINFPDDLPGSNTAAPVQETLHGCQPVTQEDGKESRISVQERQ

SEQ ID NO: 21 - protein, 476 aa, synthetic construct
  REGION 1..476: note = Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLLRWEPSSQPTIPILEVGIIAGLVLFGAVITGAVVAAVMWELKKVAKKPTNKAPHPKQEPQEINFPDDLPGSNTAAPVQETLHGCQPVTQEDGKESRISVQERQ

SEQ ID NO: 22 - protein, 509 aa, synthetic construct
  REGION 1..509: note = Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLLRWEPSSQPTIPILEVGIIAGLVLFGAVITGAVVAAVMWHMMKQIEDKIEEILSKIYHIENEIARIKKLIGEELKKVAKKPTNKAPHPKQEPQEINFPDDLPGSNTAAPVQETLHGCQPVTQEDGKESRISVQERQ

SEQ ID NO: 23 - protein, 456 aa, synthetic construct
  REGION 1..456: note = Synthetic
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLPVLSLNITCQMNKLEALVVIPIIFGILFAILLVLVFIELKKVAKKPTNKAPHPKQEPQEINFPDDLPGSNTAAPVQETLHGCQPVTQEDGKESRISVQERQ

SEQ ID NO: 24 - protein, 489 aa, synthetic construct
  REGION 1..489: note = Synthetic
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLPVLSLNITCQMNKLEALVVIPIIFGILFAILLVLVFIHMMKQIEDKIEEILSKIYHIENEIARIKKLIGEELKKVAKKPTNKAPHPKQEPQEINFPDDLPGSNTAAPVQETLHGCQPVTQEDGKESRISVQERQ

SEQ ID NO: 25 - protein, 458 aa, synthetic construct
  REGION 1..458: note = Synthetic
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLPVLSLNITCQMNKLEVGIIAGLVLFGAVITGAVVAAVMWELKKVAKKPTNKAPHPKQEPQEINFPDDLPGSNTAAPVQETLHGCQPVTQEDGKESRISVQERQ

SEQ ID NO: 26 - protein, 491 aa, synthetic construct
  REGION 1..491: note = Synthetic
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLPVLSLNITCQMNKLEVGIIAGLVLFGAVITGAVVAAVMWHMMKQIEDKIEEILSKIYHIENEIARIKKLIGEELKKVAKKPTNKAPHPKQEPQEINFPDDLPGSNTAAPVQETLHGCQPVTQEDGKESRISVQERQ

SEQ ID NO: 27 - protein, 456 aa, synthetic construct
  REGION 1..456: note = Synthetic
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLLRWEPSSQPTIPILEALVVIPIIFGILFAILLVLVFIELKKVAKKPTNKAPHPKQEPQEINFPDDLPGSNTAAPVQETLHGCQPVTQEDGKESRISVQERQ

SEQ ID NO: 28 - protein, 489 aa, synthetic construct
  REGION 1..489: note = Synthetic
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLLRWEPSSQPTIPILEALVVIPIIFGILFAILLVLVFIHMMKQIEDKIEEILSKIYHIENEIARIKKLIGEELKKVAKKPTNKAPHPKQEPQEINFPDDLPGSNTAAPVQETLHGCQPVTQEDGKESRISVQERQ

SEQ ID NO: 29 - protein, 458 aa, synthetic construct
  REGION 1..458: note = Synthetic
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLLRWEPSSQPTIPILEVGIIAGLVLFGAVITGAVVAAVMWELKKVAKKPTNKAPHPKQEPQEINFPDDLPGSNTAAPVQETLHGCQPVTQEDGKESRISVQERQ

SEQ ID NO: 30 - protein, 491 aa, synthetic construct
  REGION 1..491: note = Synthetic
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLLRWEPSSQPTIPILEVGIIAGLVLFGAVITGAVVAAVMWHMMKQIEDKIEEILSKIYHIENEIARIKKLIGEELKKVAKKPTNKAPHPKQEPQEINFPDDLPGSNTAAPVQETLHGCQPVTQEDGKESRISVQERQ

SEQ ID NO: 31 - protein, 35 aa, synthetic construct
  REGION 1..35: note = Synthetic
HMMKQIEDKIEEILSKIYHIENEIARIKKLIGEEL

SEQ ID NO: 32 - DNA, 1465 bp, synthetic construct
  misc_feature 1..1465: note = Synthetic
  source 1..1465: mol_type = other DNA
ctaagctttctagaggatccgccaccatgcacagctcagcactgctctgttgcctggtcctcctgactggggtgagggccagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgcctgtgctgagtttgaatatcacctgtcagatgaataagctcgaggccctggtggtgatccccatcatcttcggaatcctgtttgccatcctcttggtgctggtctttatcgagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacagtgagcggccgcgaattc

SEQ ID NO: 33 - DNA, 1564 bp, synthetic construct
  misc_feature 1..1564: note = Synthetic
  source 1..1564: mol_type = other DNA
ctaagctttctagaggatccgccaccatgcacagctcagcactgctctgttgcctggtcctcctgactggggtgagggccagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgcctgtgctgagtttgaatatcacctgtcagatgaataagctcgaggccctggtggtgatccccatcatcttcggaatcctgtttgccatcctcttggtgctggtctttatccatatgatgaaacagatcgaagataaaatcgaagaaatcttaagcaaaatctatcatatcgaaaacgaaatcgcgcgcatcaaaaagctgatcggcgaagagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacagtgagcggccgcgaattc

SEQ ID NO: 34 - DNA, 1471 bp, synthetic construct
  misc_feature 1..1471: note = Synthetic
  source 1..1471: mol_type = other DNA
ctaagctttctagaggatccgccaccatgcacagctcagcactgctctgttgcctggtcctcctgactggggtgagggccagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgcctgtgctgagtttgaatatcacctgtcagatgaataagctcgaggtgggcatcattgctggcctggttctctttggagctgtgatcactggagctgtggtcgctgctgtgatgtgggagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacagtgagcggccgcgaattc

SEQ ID NO: 35 - DNA, 1570 bp, synthetic construct
  misc_feature 1..1570: note = Synthetic
  source 1..1570: mol_type = other DNA
ctaagctttctagaggatccgccaccatgcacagctcagcactgctctgttgcctggtcctcctgactggggtgagggccagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgcctgtgctgagtttgaatatcacctgtcagatgaataagctcgaggtgggcatcattgctggcctggttctctttggagctgtgatcactggagctgtggtcgctgctgtgatgtggcatatgatgaaacagatcgaagataaaatcgaagaaatcttaagcaaaatctatcatatcgaaaacgaaatcgcgcgcatcaaaaagctgatcggcgaagagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacagtgagcggccgcgaattc

SEQ ID NO: 36 - DNA, 1465 bp, synthetic construct
  misc_feature 1..1465: note = Synthetic
  source 1..1465: mol_type = other DNA
ctaagctttctagaggatccgccaccatgcacagctcagcactgctctgttgcctggtcctcctgactggggtgagggccagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgctgagatgggagccgtcttcccagcccaccatccccatcctcgaggccctggtggtgatccccatcatcttcggaatcctgtttgccatcctcttggtgctggtctttatcgagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacagtgagcggccgcgaattc

SEQ ID NO: 37 - DNA, 1564 bp, synthetic construct
  misc_feature 1..1564: note = Synthetic
  source 1..1564: mol_type = other DNA
ctaagctttctagaggatccgccaccatgcacagctcagcactgctctgttgcctggtcctcctgactggggtgagggccagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgctgagatgggagccgtcttcccagcccaccatccccatcctcgaggccctggtggtgatccccatcatcttcggaatcctgtttgccatcctcttggtgctggtctttatccatatgatgaaacagatcgaagataaaatcgaagaaatcttaagcaaaatctatcatatcgaaaacgaaatcgcgcgcatcaaaaagctgatcggcgaagagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacagtgagcggccgcgaattc

SEQ ID NO: 38 - DNA, 1471 bp, synthetic construct
  misc_feature 1..1471: note = Synthetic
  source 1..1471: mol_type = other DNA
ctaagctttctagaggatccgccaccatgcacagctcagcactgctctgttgcctggtcctcctgactggggtgagggccagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgctgagatgggagccgtcttcccagcccaccatccccatcctcgaggtgggcatcattgctggcctggttctctttggagctgtgatcactggagctgtggtcgctgctgtgatgtgggagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacagtgagcggccgcgaattc

SEQ ID NO: 39 - DNA, 1570 bp, synthetic construct
  misc_feature 1..1570: note = Synthetic
  source 1..1570: mol_type = other DNA
ctaagctttctagaggatccgccaccatgcacagctcagcactgctctgttgcctggtcctcctgactggggtgagggccagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgctgagatgggagccgtcttcccagcccaccatccccatcctcgaggtgggcatcattgctggcctggttctctttggagctgtgatcactggagctgtggtcgctgctgtgatgtggcatatgatgaaacagatcgaagataaaatcgaagaaatcttaagcaaaatctatcatatcgaaaacgaaatcgcgcgcatcaaaaagctgatcggcgaagagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacagtgagcggccgcgaattc

SEQ ID NO: 40 - DNA, 1368 bp, synthetic construct
  misc_feature 1..1368: note = Synthetic
  source 1..1368: mol_type = other DNA
agcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgcctgtgctgagtttgaatatcacctgtcagatgaataagctcgaggccctggtggtgatccccatcatcttcggaatcctgtttgccatcctcttggtgctggtctttatcgagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacag

SEQ ID NO: 41 - DNA, 1467 bp, synthetic construct
  misc_feature 1..1467: note = Synthetic
  source 1..1467: mol_type = other DNA
agcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgcctgtgctgagtttgaatatcacctgtcagatgaataagctcgaggccctggtggtgatccccatcatcttcggaatcctgtttgccatcctcttggtgctggtctttatccatatgatgaaacagatcgaagataaaatcgaagaaatcttaagcaaaatctatcatatcgaaaacgaaatcgcgcgcatcaaaaagctgatcggcgaagagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacag

SEQ ID NO: 42 - DNA, 1374 bp, synthetic construct
  misc_feature 1..1374: note = Synthetic
  source 1..1374: mol_type = other DNA
agcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgcctgtgctgagtttgaatatcacctgtcagatgaataagctcgaggtgggcatcattgctggcctggttctctttggagctgtgatcactggagctgtggtcgctgctgtgatgtgggagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacag

SEQ ID NO: 43 - DNA, 1473 bp, synthetic construct
  misc_feature 1..1473: note = Synthetic
  source 1..1473: mol_type = other DNA
agcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgcctgtgctgagtttgaatatcacctgtcagatgaataagctcgaggtgggcatcattgctggcctggttctctttggagctgtgatcactggagctgtggtcgctgctgtgatgtggcatatgatgaaacagatcgaagataaaatcgaagaaatcttaagcaaaatctatcatatcgaaaacgaaatcgcgcgcatcaaaaagctgatcggcgaagagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacag

SEQ ID NO: 44 - DNA, 1368 bp, synthetic construct
  misc_feature 1..1368: note = Synthetic
  source 1..1368: mol_type = other DNA
agcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgctgagatgggagccgtcttcccagcccaccatccccatcctcgaggccctggtggtgatccccatcatcttcggaatcctgtttgccatcctcttggtgctggtctttatcgagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacag

SEQ ID NO: 45 - DNA, 1467 bp, synthetic construct
  misc_feature 1..1467: note = Synthetic
  source 1..1467: mol_type = other DNA
agcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgctgagatgggagccgtcttcccagcccaccatccccatcctcgaggccctggtggtgatccccatcatcttcggaatcctgtttgccatcctcttggtgctggtctttatccatatgatgaaacagatcgaagataaaatcgaagaaatcttaagcaaaatctatcatatcgaaaacgaaatcgcgcgcatcaaaaagctgatcggcgaagagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacag

SEQ ID NO: 46 - DNA, 1374 bp, synthetic construct
  misc_feature 1..1374: note = Synthetic
  source 1..1374: mol_type = other DNA
agcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgctgagatgggagccgtcttcccagcccaccatccccatcctcgaggtgggcatcattgctggcctggttctctttggagctgtgatcactggagctgtggtcgctgctgtgatgtgggagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacag

SEQ ID NO: 47 - DNA, 1473 bp, synthetic construct
  misc_feature 1..1473: note = Synthetic
  source 1..1473: mol_type = other DNA
agcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgctgagatgggagccgtcttcccagcccaccatccccatcctcgaggtgggcatcattgctggcctggttctctttggagctgtgatcactggagctgtggtcgctgctgtgatgtggcatatgatgaaacagatcgaagataaaatcgaagaaatcttaagcaaaatctatcatatcgaaaacgaaatcgcgcgcatcaaaaagctgatcggcgaagagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacag

SEQ ID NO: 48 - DNA, 480 bp, synthetic construct
  misc_feature 1..480: note = Synthetic
  source 1..480: mol_type = other DNA
agcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaac

SEQ ID NO: 49 - DNA, 54 bp, synthetic construct
  misc_feature 1..54: note = Synthetic
  source 1..54: mol_type = other DNA
atgcacagctcagcactgctctgttgcctggtcctcctgactggggtgagggcc

SEQ ID NO: 50 - protein, 5 aa, synthetic construct
  REGION 1..5: note = Synthetic
GGGGS

SEQ ID NO: 51 - protein, 32 aa, synthetic construct
  REGION 1..32: note = Synthetic
GGGGSGGGGSGGGGSGGGGSGGGGSGGGGSAS

SEQ ID NO: 52 - DNA, 39 bp, synthetic construct
  misc_feature 1..39: note = Synthetic
  source 1..39: mol_type = other DNA
ggaggtggcgggtccggaggtggctccggaggtggctcc

SEQ ID NO: 53 - DNA, 96 bp, synthetic construct
  misc_feature 1..96: note = Synthetic
  source 1..96: mol_type = other DNA
ggaggtggcgggtccggaggtggcgggtccggaggtggcgggtccggaggtggcgggtccggaggtggcgggtccggaggtggcgggtccgcctcc

SEQ ID NO: 54 - DNA, 54 bp, synthetic construct
  misc_feature 1..54: note = Synthetic
  source 1..54: mol_type = other DNA
ggcagcacttcgggcagtggtaagcccggaagtggtgagggtagcactaagggt

SEQ ID NO: 55 - DNA, 1014 bp, synthetic construct
  misc_feature 1..1014: note = Synthetic
  source 1..1014: mol_type = other DNA
agcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaac

SEQ ID NO: 56 - DNA, 1068 bp, synthetic construct
  misc_feature 1..1068: note = Synthetic
  source 1..1068: mol_type = other DNA
atgcacagctcagcactgctctgttgcctggtcctcctgactggggtgagggccagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaac

SEQ ID NO: 57 - DNA, 72 bp, synthetic construct
  misc_feature 1..72: note = Synthetic
  source 1..72: mol_type = other DNA
gtgggcatcattgctggcctggttctctttggagctgtgatcactggagctgtggtcgctgctgtgatgtgg

SEQ ID NO: 58 - DNA, 66 bp, synthetic construct
  misc_feature 1..66: note = Synthetic
  source 1..66: mol_type = other DNA
gccctggtggtgatccccatcatcttcggaatcctgtttgccatcctcttggtgctggtctttatc

SEQ ID NO: 59 - DNA, 24 bp, synthetic construct
  misc_feature 1..24: note = Synthetic
  source 1..24: mol_type = other DNA
tcttcccagcccaccatccccatc

SEQ ID NO: 60 - DNA, 39 bp, synthetic construct
  misc_feature 1..39: note = Synthetic
  source 1..39: mol_type = other DNA
ctgagatgggagccgtcttcccagcccaccatccccatc

SEQ ID NO: 61 - DNA, 39 bp, synthetic construct
  misc_feature 1..39: note = Synthetic
  source 1..39: mol_type = other DNA
cctgtgctgagtttgaatatcacctgtcagatgaataag

SEQ ID NO: 62 - DNA, 1319 bp, synthetic construct
  misc_feature 1..1319: note = Synthetic
  source 1..1319: mol_type = other DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagtacttcgggcagtggtaagcccgggagtggtgagggtagtactaagggtagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggaggtggcggatccggaggtggctccggaggtggctcctcgagccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtgagcggccgcaagctt

SEQ ID NO: 63 - protein, 389 aa, synthetic construct
  REGION 1..389: note = Synthetic
  SITE 389: note = misc_feature - Xaa can be any naturally occurring amino acid
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLPVLSLNITCQMNKLEX

SEQ ID NO: 64 - DNA, 1196 bp, synthetic construct
  misc_feature 1..1196: note = Synthetic
  source 1..1196: mol_type = other DNA
gcatgcctaagctttctagaggatccgccaccatgcacagctcagcactgctctgttgcctggtcctcctgactggggtgagggccagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgcctgtgctgagtttgaatatcacctgtcagatgaataagctcgag

SEQ ID NO: 65 - protein, 389 aa, synthetic construct
  REGION 1..389: note = Synthetic
  SITE 389: note = misc_feature - Xaa can be any naturally occurring amino acid
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSTSGSGKPGSGEGSTKGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNPGGGGGSGGGSGGGSQLLRWEPSSQPTIPILEX

SEQ ID NO: 66 - DNA, 1196 bp, synthetic construct
  misc_feature 1..1196: note = Synthetic
  source 1..1196: mol_type = other DNA
gcatgcctaagctttctagaggatccgccaccatgcacagctcagcactgctctgttgcctggtcctcctgactggggtgagggccagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggcagcacttcgggcagtggtaagcccggaagtggtgagggtagcactaagggtagcccaggccagggcacccagtctgagaacagctgcacccacttcccaggcaacctgcctaacatgcttcgtgatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcaccgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaaccccgggggaggtggcgggtccggaggtggctccggaggtggctcccaattgctgagatgggagccgtcttcccagcccaccatccccatcctcgag

SEQ ID NO: 67 - DNA, 189 bp, synthetic construct
  misc_feature 1..189: note = Synthetic
  source 1..189: mol_type = other DNA
aaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacagtga

SEQ ID NO: 68 - protein, 33 aa, synthetic construct
  REGION 1..33: note = Synthetic
MKQIEDKIEEILSKIYHIENEIARIKKLIGEEL

SEQ ID NO: 69 - DNA, 105 bp, synthetic construct
  misc_feature 1..105: note = Synthetic
  source 1..105: mol_type = other DNA
catatgatgaaacagatcgaagataaaatcgaagaaatcttaagcaaaatctatcatatcgaaaacgaaatcgcgcgcatcaaaaagctgatcggcgaagagctc

SEQ ID NO: 70 - DNA, 99 bp, synthetic construct
  misc_feature 1..99: note = Synthetic
  source 1..99: mol_type = other DNA
atgaaacagatcgaagataaaatcgaagaaatcttaagcaaaatctatcatatcgaaaacgaaatcgcgcgcatcaaaaagctgatcggcgaagagctc

SEQ ID NO: 71 - DNA, 189 bp, synthetic construct; identical to SEQ ID NO: 67
  misc_feature 1..189: note = Synthetic
  source 1..189: mol_type = other DNA

SEQ ID NO: 72 - protein, 95 aa, synthetic construct
  REGION 1..95: note = Synthetic
MKQIEDKIEEILSKIYHIENEIARIKKLIGEELKKVAKKPTNKAPHPKQEPQEINFPDDLPGSNTAAPVQETLHGCQPVTQEDGKESRISVQERQ

SEQ ID NO: 73 - DNA, 288 bp, synthetic construct
  misc_feature 1..288: note = Synthetic
  source 1..288: mol_type = other DNA
atgaaacagatcgaagataaaatcgaagaaatcttaagcaaaatctatcatatcgaaaacgaaatcgcgcgcatcaaaaagctgatcggcgaagagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacagtga

SEQ ID NO: 74 - protein, 88 aa, synthetic construct
  REGION 1..88: note = Synthetic
LEALVVIPIIFGILFAILLVLVFIELKKVAKKPTNKAPHPKQEPQEINFPDDLPGSNTAAPVQETLHGCQPVTQEDGKESRISVQERQ

SEQ ID NO: 75 - DNA, 264 bp, synthetic construct
  misc_feature 1..264: note = Synthetic
  source 1..264: mol_type = other DNA
ctcgaggccctggtggtgatccccatcatcttcggaatcctgtttgccatcctcttggtgctggtctttatcgagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacag

SEQ ID NO: 76 - protein, 121 aa, synthetic construct
  REGION 1..121: note = Synthetic
LEALVVIPIIFGILFAILLVLVFIHMMKQIEDKIEEILSKIYHIENEIARIKKLIGEELKKVAKKPTNKAPHPKQEPQEINFPDDLPGSNTAAPVQETLHGCQPVTQEDGKESRISVQERQ

SEQ ID NO: 77 - DNA, 363 bp, synthetic construct
  misc_feature 1..363: note = Synthetic
  source 1..363: mol_type = other DNA
ctcgaggccctggtggtgatccccatcatcttcggaatcctgtttgccatcctcttggtgctggtctttatccatatgatgaaacagatcgaagataaaatcgaagaaatcttaagcaaaatctatcatatcgaaaacgaaatcgcgcgcatcaaaaagctgatcggcgaagagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacag

SEQ ID NO: 78 - protein, 90 aa, synthetic construct
  REGION 1..90: note = Synthetic
LEVGIIAGLVLFGAVITGAVVAAVMWELKKVAKKPTNKAPHPKQEPQEINFPDDLPGSNTAAPVQETLHGCQPVTQEDGKESRISVQERQ

SEQ ID NO: 79 - DNA, 270 bp, synthetic construct
  misc_feature 1..270: note = Synthetic
  source 1..270: mol_type = other DNA
ctcgaggtgggcatcattgctggcctggttctctttggagctgtgatcactggagctgtggtcgctgctgtgatgtgggagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacag

SEQ ID NO: 80 - protein, 123 aa, synthetic construct
  REGION 1..123: note = Synthetic
LEVGIIAGLVLFGAVITGAVVAAVMWHMMKQIEDKIEEILSKIYHIENEIARIKKLIGEELKKVAKKPTNKAPHPKQEPQEINFPDDLPGSNTAAPVQETLHGCQPVTQEDGKESRISVQERQ

SEQ ID NO: 81 - DNA, 369 bp, synthetic construct
  misc_feature 1..369: note = Synthetic
  source 1..369: mol_type = other DNA
ctcgaggtgggcatcattgctggcctggttctctttggagctgtgatcactggagctgtggtcgctgctgtgatgtggcatatgatgaaacagatcgaagataaaatcgaagaaatcttaagcaaaatctatcatatcgaaaacgaaatcgcgcgcatcaaaaagctgatcggcgaagagctcaaaaaggtggccaagaagccaaccaataaggccccccaccccaagcaggaaccccaggagatcaattttcccgacgatcttcctggctccaacactgctgctccagtgcaggagactttacatggatgccaacccgtcacccaggaggatggcaaagagagtcgcatctcagtgcaggagagacag

SEQ ID NO: 82 - protein, 19 aa, synthetic construct
  REGION 1..19: note = Synthetic
MAWTVLLLGLLSHCTGSVT

SEQ ID NO: 83 - DNA, 19 bp, synthetic construct
  misc_feature 1..19: note = Synthetic
  source 1..19: mol_type = other DNA
tatccagccctcactcctt

SEQ ID NO: 84 - DNA, 22 bp, synthetic construct
  misc_feature 1..22: note = Synthetic
  source 1..22: mol_type = other DNA
cctcttggtgctggtctttatc

SEQ ID NO: 85 - DNA, 18 bp, synthetic construct
  misc_feature 1..18: note = Synthetic
  source 1..18: mol_type = other DNA
attggaacgctgcaggaa

SEQ ID NO: 86 - DNA, 20 bp, synthetic construct
  misc_feature 1..20: note = Synthetic
  source 1..20: mol_type = other DNA
agtggtgagggtagcactaa

SEQ ID NO: 87 - DNA, 18 bp, synthetic construct
  misc_feature 1..18: note = Synthetic
  source 1..18: mol_type = other DNA
attgctggcctggttctc

SEQ ID NO: 88 - DNA, 22 bp, synthetic construct
  misc_feature 1..22: note = Synthetic
  source 1..22: mol_type = other DNA
ctcgtttagtgaaccgtcagat